(19) United States
(12) Patent Application Publication
(10) Pub. No.: US 2024/0023882 A1
(43) Pub. Date: Jan. 25, 2024

(54) HYDRATION SENSORS FOR MONITORING AND DIAGNOSIS OF SKIN DISEASES IN ANY ENVIRONMENT

(71) Applicant: NORTHWESTERN UNIVERSITY, Evanston, IL (US)

(72) Inventors: John A. Rogers, Wilmette, IL (US); Anthony R. Banks, Evanston, IL (US); Surabhi Rao Madhvapathy, Evanston, IL (US); Heling Wang, Evanston, IL (US); Yonggang Huang, Evanston, IL (US); Shuai Xu, Chicago, IL (US)

(21) Appl. No.: 18/031,389

(22) PCT Filed: Oct. 18, 2021
(86) PCT No.: PCT/US2021/055357

§ 371 Date: Apr. 12, 2023

Related U.S. Application Data
(63) Continuation-in-part of application No. PCT/US2021/036765, filed on Jun. 10, 2021.
Application No. PCT/US2021/055357 is a continuation-in-part of application No. 17/043,161, filed on Sep. 29, 2020.
(60) Provisional application No. 63/092,555, filed on Oct. 16, 2020.
(60) Provisional application No. 63/037,092, filed on Jun. 10, 2020.
(60) Provisional application No. 62/650,826, filed on Mar. 30, 2018.
(60) Provisional application No. 62/696,685, filed on Jul. 11, 2018.
(60) Provisional application No. 62/791,390, filed on Jan. 11, 2019.

(51) Int. Cl. A61B 5/00; A61B 5/145; G01N 25/56

(52) U.S. Cl. CPC A61B 5/443; A61B 5/4875; A61B 5/6832; A61B 5/14521; A61B 5/002; G01N 25/56; A61B 2562/164; A61B 2562/166; A61B 2562/0271

(57) ABSTRACT

This invention relates to a soft, battery-free, flexible, non-invasive, reusable hydration sensor adherable to even small-areas and curvilinear surfaces of a body. The hydration sensor measures volumetric water content in skin as a function of depth, and wirelessly transmits data to a portable smart device. The hydration sensor includes a top layer for thermal, chemical and mechanical isolation of the hydration sensor from an environment; a bottom layer operably placed on a target area of interest on the skin; and a flexible printed circuit board (f-PCB) disposed between the top layer and the bottom layer. The f-PCB contains electronics for sensing and wireless communication. The bottom layer operably serves as a direct interface between the f-PCB and the skin and comprises a flexible adhesive for attaching the hydration sensor to the skin.

### CROSS-REFERENCE TO RELATED PATENT APPLICATIONS

This application claims priority to and the benefit of U.S. Provisional Application Ser. No. 63/092,555, filed Oct. 16, 2020.

This application is also a continuation-in-part application of PCT Patent Application Serial No. PCT/US2021/036765, filed Jun. 10, 2021, which itself claims priority to and the benefit of U.S. Provisional Patent Application Ser. No. 63/037,092, filed Jun. 10, 2020.

This application is also a continuation-in-part application of U.S. patent application Ser. No. 17/043,161, filed Sep. 29, 2020, which is a national stage entry of PCT Patent Application Serial No. PCT/US2019/025031, filed Mar. 29, 2019, which itself claims priority to and the benefit of U.S. Provisional Patent Application Ser. No. 62/650,826, filed Mar. 30, 2018, 62/696,685 filed Jul. 11, 2018, and 62/791,390, filed Jan. 11, 2019.

Each of the above-identified applications is incorporated herein by reference in its entirety.

### STATEMENT AS TO RIGHTS UNDER FEDERALLY-SPONSORED RESEARCH

This invention was made with government support under 1635443 awarded by the National Science Foundation. The government has certain rights in the invention.

### FIELD OF THE INVENTION

The present invention relates generally to biosensors, and more particularly to reliable, low-cost, fully-integrated hydration sensors for monitoring and diagnosis of skin diseases in any environment, fabricating methods and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Skin disease affects 1 in 4 individuals at a total cost of US $75 billion in yearly spending in the U.S. However, misdiagnosis rates range between less than 10% for dermatologists to 50-80% amongst general practitioners, the latter of whom receive the most patient visits related to skin conditions. Visual identification of skin diseases is non-ideal due to the indistinct nature of disease appearance. Common symptoms for various skin diseases such as inflammation, erythema, and edema are unspecific and visual tracking of symptom severity is difficult. The inverse scenario is also true: a particular disease, such as atopic dermatitis or eczema, may have several sub-categories that present different visual cues. Therefore, solely relying on lesion morphology is insufficient to determine the state of sub-surface layers of skin, particularly in individuals with skin of color, such as the type and extent of damage to the tissue, progression of the disease, and chronicity of the lesion. Clinical tools and procedures used for diagnosis such as biopsies, dermoscopy, magnetic resonance imaging (MRI), and corneometry are expensive, often not widely accessible, and typically uncomfortable for sensitive regions of the skin. The measurements can be time-consuming and/or error prone. These circumstances motivate the need for efficient and intuitive tools to monitor skin health compatible with use both inside and outside the clinic, in any environment and on nearly any location on the body surface.

Thermal sensing is a novel and non-invasive approach to monitoring skin health. Unlike traditional electrical impedance-based techniques (corneometry), these sensors can measure the thermal properties of skin as a function of depth up to several millimeters. Skin thermal sensors demonstrated in past literature involve some combination of limiting drawbacks, including (i) shallow measurement depth (<a few 100 μm), (ii) requirement of expensive cleanroom micro-fabrication, (iii) external power source/battery-reliant operation, (iv) absence of theoretical frameworks to provide direct connections between the measured data and clinically relevant information like skin water content, (v) large measurement errors (±12%) and poor repeatability, (vi) lack of smartphone integration/convenient sensor readout capabilities and/or most importantly (vii) lack of clinical validation across a range of important skin disorders and conditions, inside and outside of hospital facilities.

Therefore, a heretofore unaddressed need exists in the art to address the aforementioned deficiencies and inadequacies.

### SUMMARY OF THE INVENTION

This invention in one aspect discloses a hydration sensor for monitoring and/or diagnosing a skin condition, and characterizing the hydration status in healthy individuals, for cosmetic and health/wellness applications. The hydration sensor includes a top layer for thermal, chemical and mechanical isolation of the hydration sensor from the environment; a bottom layer operably placed on a target area of interest on the skin; and a flexible printed circuit board (f-PCB) disposed between the top layer and the bottom layer. The f-PCB contains electronics for sensing and wireless communication. The bottom layer operably serves as a direct interface between the f-PCB and the skin. The top layer serves to thermally, chemically and mechanically isolate the underlying layers and electronics from the environment.

In one embodiment, the electronics comprises a heating circuit comprising a heating element for operably heating the target area of interest of the skin; and a sensing circuit comprising a temperature sensor for simultaneously recording a transient temperature change (ΔT) thereof.

In one embodiment, the heating element comprises a heater comprising at least one resistor.

In one embodiment, the heater comprises two or more surface-mount (SMT) thin film resistors, thick film resistors, through-hole resistors, and/or ultrathin-film (about 50-200 nm thick) metal resistors connected in series to form a heater.

In one embodiment, the temperature sensor comprises at least one SMT negative temperature coefficient thermistor, positive temperature coefficient thermistor, resistance temperature detector (RTD), thermocouple or any other conductive temperature sensor.

In one embodiment, the heating element and the temperature sensor are arranged from each other by a distance.

In one embodiment, the distance is determined by the design requirement of depth sensitivity into the skin, and ranges from about 10 μm to about 10 mm.

In one embodiment, the heater and temperature sensor are the same component, where the distance between them is zero.

In one embodiment, the sensing circuit further comprises a microcontroller (μC).

In one embodiment, the heater is operably switchable between ON and OFF controlled by the microcontroller.

In one embodiment, the microcontroller is programmable with custom-designed embedded codes using at least one of near field communication (NFC), Wi-Fi/Internet, Bluetooth, Bluetooth low energy (BLE), and GSM/Cellular communication protocols for wireless communication of the hydration sensor to a custom smartphone application.

In one embodiment, the electronics further comprises a primary antenna tuned to primary frequency, and a secondary antenna.

In one embodiment, the primary antenna is a transmission coil of an external device that is capable of wireless communications using the NFC protocol.

In one embodiment, the data transfer/powering strategy can be translated easily to various wireless techniques, e.g., Wi-fi, Bluetooth/BLE, cellular network, and so on.

In one embodiment, the external device is a smartphone, a tablet, a computer, or any electronic device with data reading capability.

In one embodiment, the secondary antenna comprises a first antenna electronically connected for powering the sensing circuit and for communicating data to the external device using the NFC protocol, and a second antenna electronically connected to the heating circuit for powering the heating element.

In one embodiment, the first antenna and the second antenna are arranged in a concentric geometry.

In one embodiment, the first antenna has a quality-factor (Q) that is relatively high to enable good communication distance and coupling across external devices with different primary antennae, and the second antenna has the quality-factor that is relatively low to support adequate power harvesting despite the difference between its resonance frequency and that of the primary coil.

In one embodiment, the quality-factor of the first antenna is about 11, and the quality-factor of the second antenna is about 8.

In one embodiment, the first antenna is tuned to the primary frequency, and the second antenna is tuned to a secondary frequency that is different from the primary frequency so as to prevent interference with the first antenna.

In one embodiment, the primary frequency is a standard NFC frequency of about 13.56 MHz, and the secondary frequency is about 19.04 MHz.

In one embodiment, the hydration sensor can be operated with a single antenna.

In one embodiment, the hydration sensor has a dynamic temperature range of about 18-45° C., adjustable through an amplifier gain, with a minimum resolution of about 15 mK, limited by the ADC.

In one embodiment, the electronics comprises a generic design including blocks of (a) a powering system comprising voltage/power regulators driven by an external battery or magnetic induction to supply power to the heater, sensing and communication circuits; (b) an ADC chip or data modulator to prepare the output of the sensing circuit for transmission to an external readout device; and (c) a transceiver chip having at least one of Wi-Fi, Bluetooth, BLE, NFC, and GSM/Cellular communication protocols to transmit the output signal to the external readout device.

In one embodiment, the f-PCB is formed of a flexible material.

In one embodiment, the flexible material comprises polyimide (PI) polyethylene terephthalate (PET), or any one of them in combination with a stiff PCB material including FR-4 and thin layers of copper/metal interconnects forming the circuitry.

In one embodiment, the f-PCB comprises open spaces and/or mechanical relief cuts for enhancing the overall flexibility, and limiting lateral thermal transport through the PI, away from the sensing components.

In one embodiment, the metal interconnect trace width is minimized to limit heat sinking away from the sensing area of the hydration sensor, thereby improving the sensitivity.

In one embodiment, the bottom layer comprises a flexible adhesive layer bonding to a thin layer of SiO₂ sputter-coated on a backside of the f-PCB.

In one embodiment, the bottom layer adheres to the f-PCB through use of silicone bonding material, epoxy, glue, or commercial adhesive.

In one embodiment, the bottom layer further comprises an ultrathin fabric of fiberglass or a reinforcement material embedded in the flexible adhesive layer for enhancing the mechanical robustness of the hydration sensor.

In one embodiment, the reinforcement material is flexible and has varying mesh density and thickness to lend tear resistance to the bottom layer.

In one embodiment, the flexible adhesive layer is formed of silicone or silicone gel, or commercially available double-sided skin-safe adhesives (preferably hypoallergenic), with the ratio of silicone and silicone gel being adjusted to co-optimize mechanical integrity and tackiness of the adhesive.

In one embodiment, the relative ratio of silicone and silicone gel in the mixture to form the flexible adhesive is co-optimized for mechanical integrity of the adhesive and level of tackiness of the adhesive.

In one embodiment, the top layer is a shell-like top encapsulation layer including small air gaps for thermally insulating the critical sensing components.

In one embodiment, the top layer is formed of a flexible material including silicone or silicone gel.

In one embodiment, the top layer comprises an air pocket/gap/shell which thermally isolates the sensing area of the hydration sensor from the environment, thereby improving hydration sensor sensitivity; a shell made of silicone or similar materials, preferably hypoallergenic, to chemically and mechanically isolate and protect the underlying layers of the hydration sensor from external elements including water, dust, and/or from user touch; or a hollow shell-like structure to provide a soft touch or feel to the user.

In one embodiment, the top layer is fabricated using molds to cure the silicone or similar materials, preferably hypoallergenic.

In one embodiment, the top layer is formed of a flexible material including silicone or silicone gel, low/high density polyethylene (LDPE/HDPE), polystyrene, Teflon®, and various other flexible polymers.

In one embodiment, the hydration sensor has low flexural rigidity and effective modulus, to facilitate application even on highly curved features of the skin.

In one embodiment, the hydration sensor is compatible for use in conjunction with other adhesives/tapes/bandages for example Tegaderm™, Scotch Tape™, surgical tape, for applications on highly curved features on the human body/skin.

In one embodiment, the hydration sensor is a soft, thin, wireless, and battery-free skin hydration sensor.

In one embodiment, the hydration sensor can be used for applications to measure thermal conductivity, thermal diffusivity, heat capacity and other thermal properties of any material as a function of depth.

In one embodiment, the hydration sensor can be used for applications to measure water content of any material surface as a function of depth, including hydrogels, plants (irrigation and agriculture applications), food preservation (dried food products, grains, fruits, meats), and/or concrete (industrial applications).

In one embodiment, the hydration sensor can be used to monitor composition of food/beverages, medicines/industrial chemicals.

In one embodiment, the hydration sensor is compatible with alcohol-based cleaning wipes allowing for re-use across different users, without any damage to the hydration sensor or loss in efficacy of the hydration sensor adhesive.

In one embodiment, the hydration sensor can be sterilized using alcohol, autoclave steam sterilization, and gas phase sterilization.

In one embodiment, the hydration sensor is re-usable and removal without irritation to the skin or damage to the hydration sensor.

In one embodiment, the thermal properties of the skin comprise thermal conductivity (k) and thermal diffusivity (α) of the skin that are related to water content (φ) of the skin, wherein the water content is a function of a skin depth.

In one embodiment, the water content φ_{E} (epidermis) and φ_{D} (dermis) are determined from the measured temperature change ΔT vs. time t (full waveform analysis).

In one embodiment, the temperature change (ΔT) in the temperature sensor is operably captured by an ADC and transmitted to the external device.

In one embodiment, the water content φ_{E} (epidermis) and φ_{D} (dermis), and skin surface temperature T₀ are used to determine a normal state or a disease state of the skin.

In one embodiment, the water content φ_{E} (epidermis) and φ_{D} (dermis), and skin surface temperature T₀ are used to diagnose various skin diseases.

In one embodiment, the water content φ_{E} (epidermis) and φ_{D} (dermis), and skin surface temperature T₀ are serve as quantitative metrics of an efficacy of a treatment of a skin disease.

In one embodiment, the hydration sensor is usable for monitoring the skin condition, or other health and wellness products including skin moisturizers, lotions, and/or creams.

In one embodiment, an aggregate water content φ comprising the full measurement volume serves as a quantitative metric for diagnosis of skin diseases or of an efficacy of a treatment of a skin disease.

In one embodiment, the hydration sensor is usable for delivering treatment, monitoring the effects, modulating the treatment protocol as necessary, and/or potentially predicting for flares based on quantitative, individualized measurements on specific lesion sites.

In one embodiment, the hydration sensor is usable for monitoring water content of internal organs for various diseases where traditional monitoring techniques fail to offer continuous assessment of organ health. In one embodiment, the hydration sensor is usable for monitoring organs during organ transport for applications in organ transplant.

In one embodiment, the hydration sensor is usable in both clinical and at-home settings.

In one embodiment, the hydration sensor can be used to monitor hydration status of healthy skin for applications in cosmetics, sports, and health/wellness.

In one embodiment, the skin health data (hydration of dermis, epidermis and skin temperature) for both lesions/healthy sites can be shared with physicians for remote medical care applications.

In another aspect, the invention relates to a method of fabricating a hydration sensor. The method includes forming a bottom layer comprising a layered structure of a first flexible layer, a second flexible layer, and a fabric of fiberglass/a reinforcement material embedded between the first flexible layer and the second flexible layer; treating a surface of the bottom layer with ultraviolet (UV) light to create reactive —OH groups on the surface of the bottom layer; sputter coating a layer of SiO₂ onto a backside of an f-PCB, wherein the f-PCB contains electronics for sensing and wireless communication; adhering the f-PCB to the bottom layer via a covalent dehydration reaction between the —OH groups on the layer of SiO₂ sputter coated onto the backside of the f-PCB and the treated surface of the bottom layer; forming a top shell layer including small air gaps; placing the top shell layer and the bottom layer such that the f-PCB is positioned between the top shell layer and the bottom layer and subsequently curing them on a hotplate so as to seal them; and cutting the sealed structure out in a desired shape to form the hydration sensor.

In one embodiment, each of the first flexible layer and the second flexible layer is formed of silicone or silicone gel, or commercially available double-sided skin-safe adhesives (preferably hypoallergenic), with the ratio of the silicone and silicone gel being adjusted to co-optimize mechanical integrity and tackiness of the adhesive.

In one embodiment, the reinforcement material is flexible and has varying mesh density and thickness to lend tear resistance to the bottom layer.

In one embodiment, the bottom layer adheres to the f-PCB through use of silicone bonding material, epoxy, glue, or commercial adhesive.

In one embodiment, the top shell layer is formed of silicone or silicone gel, low/high density polyethylene (LDPE/HDPE), polystyrene, Teflon®, and various other flexible polymers.

In one embodiment, the top layer comprises an air pocket/gap/shell which thermally isolates the sensing area of the hydration sensor from the environment, thereby improving hydration sensor sensitivity; a shell made of silicone or similar materials, preferably hypoallergenic, to chemically and mechanically isolate and protect the underlying layers of the hydration sensor from external elements including water, dust, and/or from user touch; or a hollow shell-like structure to provide a soft touch/feel to the user.

In one embodiment, the top layer is fabricated using molds to cure the silicone or similar materials, preferably hypoallergenic.

In one embodiment, the f-PCB is formed of a flexible material comprising polyimide (PI), polyethylene terephthalate (PET), or any one of them in combination with stiff PCB material such as FR-4.

In one embodiment, the f-PCB comprises open spaces and/or mechanical relief cuts for enhancing the overall flexibility, and limiting lateral thermal transport through the PI, away from the sensing components.

In one embodiment, the electronics comprises a heating circuit comprising a heating element for operably heating the target area of interest of the skin; and a sensing circuit comprising a temperature sensor for simultaneously recording a transient temperature change (ΔT) thereof.

In one embodiment, the heating element and the temperature sensor are arranged laterally away from each other by a distance.

In one embodiment, the distance is determined by the design requirement of depth sensitivity into the skin, and ranges from about 10 μm to about 10 mm.

In one embodiment, the heater and temperature sensor are the same component, wherein the distance between them is zero.

In one embodiment, the sensing circuit further comprises a microcontroller (μC).

In one embodiment, the microcontroller is programmable with a custom-designed embedded code using an NFC read/write interface allowed for wireless communication of the hydration sensor to a custom smartphone application.

In one embodiment, the electronics further comprises a primary antenna tuned to a primary frequency, and a secondary antenna.

In one embodiment, the primary antenna is a transmission coil of an external device that is capable of wireless communications using an NFC protocol.

In one embodiment, the external device is a smartphone, a tablet, a computer, or any electronic device with data reading capability.

In one embodiment, the secondary antenna comprises a first antenna electronically connected to the microcontroller for powering the sensing circuit and for communicating data to the external device using the NFC protocol, and a second antenna electronically connected to the heating circuit for powering the heating element.

In one embodiment, the first antenna and the second antenna are arranged in a concentric geometry.

In one embodiment, the first antenna is tuned to the primary frequency, and the second antenna is tuned to a secondary frequency that is different from the primary frequency so as to prevent interference with the first antenna.

In one embodiment, the hydration sensor can be operated using a single antenna.

In one embodiment, the electronics comprises a generic design including blocks of (a) a powering system comprising voltage/power regulators driven by an external battery or magnetic induction to supply power to the heater, sensing and communication circuits; (b) an ADC (analog to digital convertor) chip or data modulator to prepare the output of the sensing circuit for transmission to an external readout device; and (c) a transceiver chip having at least one of NFC, Wi-Fi/Internet, Bluetooth, BLE, and GSM/Cellular communication protocols to transmit the output signal to the external readout device.

In yet another aspect, the invention relates to a method of monitoring and/or diagnosing a condition of a skin. The method includes attaching a hydration sensor onto a target area of interest of the skin, wherein the hydration sensor comprising an f-PCB containing electronics for sensing and wireless communication, wherein the electronics comprising a heating circuit comprising a heating element; a sensing circuit comprising a temperature sensor; a microcontroller coupled to the heating element and the sensing circuit, and a transceiver coupled to the heating element, the sensing circuit and the microcontroller; heating the target area of interest of the skin by the heating element and simultaneously recording a transient temperature change (ΔT) thereof by the temperature sensor; obtaining water content of the target area of interest of the skin from the temperature change (ΔT); and determining a condition of the skin at the target area of interest based on the obtained water content.

In one embodiment, the water content comprises water content φ_{E} of the epidermis and water content φ_{D} of the dermis.

In one embodiment, the step of obtaining the water content comprises separately determination of φ_{E} and φ_{D} from the temperature change ΔT vs. t curves, thereby enabling real-time display of φ_{E} and φ_{D} on the phone application shortly after completing the measurement.

In one embodiment, the transceiver transmits data through a wireless communication protocol including NFC, Wi-Fi/Internet, Bluetooth, BLE, or Cellular communication.

In one embodiment, the transceiver relays data to an external device through various wireless communication methods including NFC, Wi-Fi/Internet, Bluetooth, BLE, or Cellular communication.

In one embodiment, the transceiver is powered wirelessly or through use of a battery/capacitive discharge mechanism.

In one embodiment, the transceiver comprises a primary antenna tuned to primary frequency, and a secondary antenna.

In one embodiment, the primary antenna is a transmission coil of an external device that is capable of wireless communications using the NFC protocol.

In one embodiment, the secondary antenna comprises a first antenna electronically connected to the microcontroller for powering the sensing circuit and for communicating data to the external device using the NFC protocol, and a second antenna electronically connected to the heating circuit for powering the heating element.

In one embodiment, the first antenna and the second antenna are arranged in a concentric geometry.

In one embodiment, the first antenna is tuned to the primary frequency, and the second antenna is tuned to a secondary frequency that is different from the primary frequency so as to prevent interference with the first antenna.

In one embodiment, the external device is a smartphone, a tablet, a computer, or any electronic device with data reading capability.

In one embodiment, the method further comprises programming the microcontroller with a custom-designed embedded code using an NFC read/write interface allowed for wireless communication of the hydration sensor to a custom smartphone application.

In one embodiment, the step of heating the target area of interest of the skin comprises powering the heating element through a regulated DC supply at a voltage of about 3.3 V derived from the secondary AC voltage at the second antenna, resulting in a constant thermal power of q=10 mW/mm².

In one embodiment, the temperature sensor connects as one of the arms of a Wheatstone bridge powered by a rectified 2.1 V from the RF μ microcontroller, wherein the voltage across the arms of the Wheatstone bridge is amplified, subsequently read by the RF microcontroller ADC, then transmitted through the first antenna to the phone NFC reader, and recorded in the phone's memory, and displayed on the screen.

In one embodiment, the step of determining the condition of the skin at the target area of interest comprises comparing the obtained water content to a standard water content at the target area of interest so as to determine a normal state or a disease state of the skin.

In one embodiment, the step of determining the condition of the skin at the target area of interest comprises diagnosing a skin disease at the target area of interest based on wherein the obtained water content thereof.

In one embodiment, the step of determining the condition of the skin at the target area of interest comprises diagnosing a skin disease at the target area of interest by comparing measurements of skin barrier function to a clinician-derived overall dry skin (ODS) score.

In one embodiment, the step of determining the condition of the skin at the target area of interest comprises evaluating an efficacy of a treatment of the skin disease.

In one embodiment, the method further comprises one or more steps of delivering treatment, monitoring the effects, modulating the treatment protocol as necessary, and/or potentially predicting for flares based on quantitative, individualized measurements on specific lesion sites.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically soft, wireless, battery-free skin hydration sensors according to embodiments of the invention. Panel A: Diagram illustrating the layers and components of the device. Panel B: Photograph of a sensor bent between the thumb and index finger. Panel C: Photograph of the sensor mounted on a human forearm while the skin is under torsion. The sensor is secured to the skin with Tegaderm film overlaying the device. Panel D: Photograph of the sensor mounted on the shin with Tegaderm film placed over the device. Panel E: Photograph of the sensor mounted on the face without the assistance of external adhesives. Panel F: Image of the sensor mounted on the knuckle. The sensor is secured to the skin using Tegaderm film. Panel G: Image of the sensor mounted on the antecubital fossa without external adhesives and corresponding NFC readout of the sensor response displayed on a smartphone, providing a visual of the actual measurement procedure.

FIG. 2 shows device design, operation, and performance according to embodiments of the invention. Panel A: Circuit diagram of the device and wireless pairing with a smart phone. A screenshot of a completed NFC readout from the device on the smart phone. Panel B: Photograph of a fully assembled flexible printed circuit board (f-PCB) for the device. Component 1 corresponds to a 13.56 MHz antenna used for powering of the RF μC, sensing circuit, and communication, component 2 is the 19.04 MHz antenna used to deliver a constant q=10 mW/mm² of thermal power to the heater, component 3 is the voltage regulator, component 4 is the RF μC, and component 5 is the instrumentation amplifier from the circuit diagram in panel A. A microscope image of the heater and the NTC thermistor are shown in a subset of the image. The scalebar corresponds to 1 mm. Panel C: Frequency sweep of the phase angle recorded from an impedance analyzer displaying the peak resonant frequency of the two antennas. The Q factor of each antenna is noted above its corresponding resonant peak. Panel D: Root-mean-square rectified voltage from Ant. 2 recorded across several different smartphone readers. The dotted line indicates the minimum RMS voltage (V_{threshold}) for reliable operation. Panel E: IR Camera image of the heater and sensor when the heater is OFF and after the heater is ON. Panel F: Representative transient temperature (ΔT) response generated by a single sensor (n=1) on four different substrate materials, water (k=0.6 W/m-K), S170 (k=0.4 W/m-K), S184 (k=0.2 W/m-K) and air (k=0.02 W/m-K). Panel G: Box plots of ΔT at t=13 s normalized to ΔT for water at t=13 s as measured by n=6 sensors (measured 5× each) on the same four materials as in panel F.

FIG. 3 shows evaluating the skin water content, φ, according to embodiments of the invention. Panel A: Pictorial diagram of the structure of skin. The parameter h represents thickness of the epidermis. Certain important features are noted in the diagram: (1) sebaceous glands, (2) eccrine sweat glands, and (3) blood vessels. Panel B: Workflow diagram illustrating the procedure for FEA fitting of epidermal hydration level φ_{E} (%) and dermal hydration level φ_{D} (%) from the measured ΔT vs. time curve. Panel C: Cross-sectional view of the temperature distribution induced by the heater on the skin at short time (t=2 s). Panel D: Cross-sectional view of the temperature distribution induced by the heater on the skin at long time (t=13 s). Panel E: Relationship of the ΔT at short times (t=2 s) to φ_{E} and φ_{D}. Panel F: Relationship of the ΔT at long times (t=13 s) to φ_{E} and φ_{D}. Panel G: Violin plots of φ_{E} and φ_{D} for six different body locations (nₚₐₜᵢₑₙₜₛ=16). Values for φ_{D} are not shown for the heel due to the large value of h (about 600 μm).

FIG. 4 shows measurements on atopic dermatitis patients according to embodiments of the invention. φ for the epidermis (light gray bars) and dermis (dark gray bars), and photographic image of the lesion and non-lesional area for patients with chronic atopic dermatitis lesions (panels A-D) and acute atopic dermatitis lesions (panels E-H). Measurements involved n=7 patients and n=13 lesions. Dots embedded in the images represent exact measurement locations. Data for the lesions not displayed here are in FIG. 17. Gray lines between plots for φ and corresponding images demarcate different patients. Patient demographics are in Table 4. Error bars represent the standard deviations of three measurements repeated consecutively on the body location. In the absence of perilesional areas of skin with normal appearance, the contralateral location served as a comparison. Panel I: Histogram highlighting the difference between the non-lesional epidermal hydration (φ_{E, N}) and the lesional epidermal hydration (φ_{E, L}) for all n=13 lesions. Panel J: Histogram highlighting the difference between the non-lesional dermal hydration (φ_{D, N}) and the lesional dermal hydration (φ_{D, L}) for all n=13 lesions. Panel K: Histogram highlighting the difference in non-lesional skin surface temperature (T_{0, N}) and lesional skin surface temperature (T_{0,L}) for all n=13 lesions. Computed two-sided p-values using the Wilcoxon ranked sign test is displayed above each histogram.

FIG. 5 shows measurements on patients with psoriasis and urticaria according to embodiments of the invention. φ for the epidermis (light gray bars) and dermis (dark gray bars), and photographic image of the lesion and non-lesional area for patients with psoriasis (panels A-D). Measurements involved n=3 patients and n=7 lesions. Dots embedded in the images represent exact measurement locations. Data for lesions not displayed here are in FIG. 17. Gray lines between plots for φ and corresponding images demarcate different patients. Patient demographics are in Table 4. Error bars represent the standard deviations of three measurements repeated consecutively on the body location. In the absence of perilesional areas of skin with normal appearance, the contralateral location served as a comparison. Panel E: Histogram highlighting the difference between the non-lesional epidermal hydration (φ_{E, N}) and the lesional epidermal hydration (φ_{E, L}) for all n=7 lesions. Panel F: Histogram highlighting the difference between the non-lesional dermal hydration (φ_{D, N}) and the lesional dermal hydration (φ_{D, L}) for all n=7 lesions. Panel G: Histogram highlighting the difference in non-lesional skin surface temperature (T_{0, N}) and lesional skin surface temperature (T_{0,L}) for all n=7 lesions. Panels H-K: φ for the epidermis (light gray bars) and dermis (dark gray bars), and photographic image of the lesion and non-lesional area for patients with urticaria. Measurements involved n=2 patients and n=4 lesions. Gray lines between plots for φ and corresponding images demarcate different patients. Error bars represent the standard deviations of three measurements repeated consecutively on the body location. In the absence of perilesional areas of skin with normal appearance, the contralateral location served as a comparison. Panel L: Histogram highlighting the difference between the non-lesional epidermal hydration (φ_{E, N}) and the lesional epidermal hydration (φ_{E, L}) for all n=4 lesions. Panel M: Histogram highlighting the difference between the non-lesional dermal hydration (φ_{D, N}) and the lesional dermal hydration (φ_{D, L}) for all n=4 lesions. Panel N: Histogram highlighting the difference in non-lesional skin surface temperature (T_{0, N}) and lesional skin surface temperature (T_{0,L}) for all n=4 lesions. Computed two-sided p-values using the Wilcoxon ranked sign t-test is displayed above each histogram.

FIG. 6 shows the effect of moisturizer on a subject diagnosed with atopic dermatitis and xerosis cutis according to embodiments of the invention. Panels A-B: φ for the epidermis (light gray bars) and dermis (dark gray bars), of the lesion on both legs of a patient with xerosis cutis before and 30 minutes after application of moisturizing cream to the skin. Dots embedded in the images represent exact measurement locations. The region of the graph shaded in orange represents the data post-moisture. Error bars indicate measurements repeated three times consecutively on the same location. Panel C: Photograph of the lesion on the right leg of the patient corresponding to the data in panel (A) before and 30 minutes after application of moisturizer. Panel D: φ for the epidermis and dermis on the normal skin of the forehead before and 30 minutes after application of moisturizer to the skin. No lesions for this patient appeared on the forehead. Panel E: Image of the forehead of the patient before application of moisturizer. Panels F-G: φ for the epidermis and dermis and corresponding images on the atopic dermatitis lesion and non-lesional area for the same patient on three different body locations before and 30 minutes after application of moisturizer. In panels F-G, no post-moisturizer photographs were recorded.

FIG. 7 shows an encapsulation procedure according to embodiments of the invention. Step A: Schematic diagram of a bare glass slide. The same glass slide coated with (step B) a poly(methyl)methacrylate (PMMA) release layer followed by (step C) silicone gel. Step D: A piece of pre-cut fabric adhered to the silicone gel via Van der Waals forces, and (step E) an additional layer of tacky silicone gel. Step F: UV treatment of the resulting structure formed from steps A-E. Step G: Schematic image of the SHS flexible printed circuit board coated on the bottom side with SiO₂ being placed onto the bottom layer structure. Step H: An aluminum mold with silicone poured inside that forms (step I) the silicone top shell when cured. Step J: The resulting bottom layer structure with uncured silicone screen printed around the edges of the f-PCB. Step K: The top shell adhered to the bottom layer by way of the uncured, screen-printed silicone. Step L: The finished skin hydration sensor after curing the screen-printed silicone, cutting out the final structure out using a custom-made die, and peeling it off the glass slide.

FIG. 8 shows individual antenna performance and interference characteristics according to embodiments of the invention. Panel A: Schematic illustration of the NFC system. The magnetic field distributions around the Ant. 2 with Ant. 1 (panel B) and without Ant. 1 (panel C). Panel D: The inductance and Q factor versus frequency (top) and the S11 parameter of the matched ant.1 (bottom). Panel E: The inductance and Q factor versus frequency (top) and the S11 parameter of the matched Ant. 2 (bottom). Panel F: The magnitude (top) and phase (bottom) of the impedance for the ant. 2 with and without Ant. 1. Panel G: Comparison of a fully wired data acquisition system (with a current source and digital multimeter) and the wireless SHS platform on two standard materials with known k (Sylgard 184, k=0.2 W/m-K, Sylgard 170, k=0.4 W/m-K). In this case, the width of copper interconnects was 25 μm instead of the 60 μm width used throughout the rest of this work, leading to larger sensitivity relative to the other sensors. The difference in width (25 μm) resulted from in-house fabrication capabilities compared to all other devices (60 μm) which were outsourced to an external vendor. Panel H: Variation in temperature vs, ADC result of the SHS resulting in variations from the thermistor resistance. The minimum, maximum, and typical resistance tolerances are derived from the thermistor datasheet. Panel I: Variations in the temperature vs. ADC result of the SHS resulting in small variations in the rectified voltage output from the microcontroller.

FIG. 9 shows measurements of ΔT on curved surfaces according to embodiments of the invention. Panel A: Schematic diagram of a hemisphere of Sylgard 184 with radius of curvature ρ. Panel B: Measurements of ΔT as a function of time on flat Sylgard 184, followed by curved (ρ=1 cm) Sylgard 184, followed by flat Sylgard 184 once again.

FIG. 10 shows factors that influence device sensitivity according to embodiments of the invention. Panel A: Influence of measurement power on ΔT at t=13 s of measurement time for different q on two samples of PDMS with different k (Sylgard 184 (k=0.2 W/m-K) and Sylgard 170 (k=0.4 W/m-K)). The dotted line indicates a linear fit for the measured points shown as symbols. R² is provided in the figure. Panel B: ΔT measured at t=13 s and inversely determined water content (blue) from a mixture of different volume percentages of water in vegetable glycerin (measured k_{glycerin}=0.285 W/m-K and α_{glycerin}=0.093 mm²/s). The mixture of vegetable glycerin and water served as a benchtop model for the micromechanics model to determine φ. Panel C: Influence of copper trace width on the transient temperature response of ΔT for a mixture of water (k=0.6 W/m-K) and vegetable glycerin (k=0.285 W/m-K) as a function of water content (φ) at a measurement time of t=13 s and thermal power q=10 mW/mm² through the heater. Panel D: ΔT at t=13 s as a function of φ for the water-glycerin mixture with varying silicone gel adhesive thickness.

FIG. 11 shows differences in sensitivity between a separated heater-sensor design and single heater/sensor structure, according to embodiments of the invention. Panel A: Analytical solution for a simplified model. Panel B: FEA based on the actual designs.

FIG. 12 shows measurement depths according to embodiments of the invention. Panel A: ΔT vs. t for bilayer structures of Sylgard 184 (k=0.2 W/m-K) of different thickness h on 12 mm thick (bulk) Sylgard 170 (k=0.4 W/m-K). Panel B: ΔT at t=13 s for different h of S184. (C) Heat profile in skin generated by the heater at different time t for α=0.15 mm²/s.

FIG. 13 shows skin thermal properties vs. water content as determined from the micromechanics model according to embodiments of the invention. Panel A: thermal conductivity. Panel B: thermal diffusivity.

FIG. 14 shows the relationship of ΔT vs. φ_{E} and φ_{D} for a thick epidermis according to embodiments of the invention. Panel A: short time (t=2 s). Panel B: long time (t=13 s).

FIG. 15 shows Error in the determined water content due to error in measurements of ΔT according to embodiments of the invention. Panels A-B: Error in φ_{E} and φ_{D} vs. relative error in temperature for two water contents. Panel C: The maximum and average error for φ_{E} and φ_{D} in the range 5%˜95% with 3% relative error in temperature.

FIG. 16 shows FEA curve fits of experimental ΔT vs. t data for healthy/normal subjects according to embodiments of the invention. Panels A-F: Subject 1 (female). Panels G-L: Subject 14 (male). RMS error appears above each graph.

FIG. 17 shows T₀ and ScH data corresponding to the data in panels A-H of FIG. 4, respectively.

FIG. 18 shows remaining lesions according to embodiments of the invention. Panels A-E: Photographs, φ, T₀, and ScH for the remaining atopic dermatitis lesions not displayed in FIG. 4. Panels F-H: Photographs, φ, T₀, and ScH for the remaining psoriasis lesions not displayed in FIG. 5. Panels I-J: Photographs, φ, T₀, and ScH for two lesions on a patient diagnosed with rosacea on both cheeks. ScH and T₀ data appear as black circles and red squares, respectively.

FIG. 19 shows histograms for ScH and TEWL measured by gpskin on diseased populations for Panel A: Atopic dermatitis. Panel B: Psoriasis. Panel C: Urticaria, according to embodiments of the invention.

FIG. 20 shows FEA curve fits of experimental ΔT vs. t data for patients with skin diseases according to embodiments of the invention. Panels A-G: patient 23 with Psoriasis. Panels H-J: patient 21 with Atopic Dermatitis.

FIG. 21 shows T₀ and ScH data corresponding to the data in FIG. 5. Panels A-D: Correspond to the data in panels A-D of FIG. 5 and Panels E-F: correspond to panels H-K of FIG. 5. ScH and T₀ data appear as black circles and red squares, respectively.

FIG. 22 shows TEWL data for all lesions and non-lesional areas according to embodiments of the invention. Panels A-H: correspond to the data in panels A-H of FIG. 4, respectively. Panels I-P: correspond to the data in panels A-H of FIG. 5, respectively, and Panels Q-Z: correspond to the data in panels A-J of FIG. 18, respectively.

FIG. 23 shows the moisturizer treatment for an additional atopic dermatitis patient according to embodiments of the invention.

FIG. 24 shows a schematic illustration of the FEA model according to embodiments of the invention.

FIG. 25 shows the influence of the environmental temperature on a sensor response according to embodiments of the invention. Before heating, the steady-state temperature, T of the device is 35° C., which is close to body temperature. The convective heat transfer coefficient between the outer surface of the encapsulation and the environment is 10 W/(m²-K). Air has a very narrow range of thermal conductivity (about 0.024-0.027 W/m-K) for a wide range of outdoor temperature conditions (0-40° C.) and the relative humidity levels (0-100%).

FIG. 26 shows ODS scoring criteria with representative examples. The scale bar in each image indicates 5 mm. Informed consent for use of images in publication was obtained upon study enrollment.

FIG. 27 shows comparison of SHS measurements organized by anatomic location and ODS score. Locations are: (a) arm, (b) leg, and (c) forehead. The red line indicates median. The top and bottom edges of the box indicate 75th and 25th percentiles, respectively. The notch boundaries indicate 95% CI of the median. The upper and lower whisker bounds are defined as 75th percentile plus 1.5 times the interquartile range (IQR) and 25th percentile minus 1.5*IQR, respectively. Red crosses indicate outliers. Pairwise comparison from post-hoc analysis are included (ns−p>0.05, * p≤0.05, ** p≤0.01, *** p≤0.001). Measurements taken after moisturizer application in the study extension are not included.

FIG. 28 shows comparison of absolute change in SHS measurements after moisturizer application. The difference between the mean post- and pre-moisturizer measurements is plotted against ODS score evaluated prior to moisturizer application and separated by body location. Post-moisturizer measurements were taken fifteen minutes after moisturizer application to allow for absorption of the moisturizer into the skin.

FIG. 29 shows comparison of SHS measurements with healthy normal (HNL) subjects, subjects with xerosis cutis (XC), and subjects with atopic dermatitis (AD) organized by anatomic location. Locations are: (a) arm, (b) leg, and (c) forehead. Measurements were labeled ‘HNL’ if the corresponding ODS score was 0 and ‘XC’ if the corresponding ODS score was ≥1. Measurements from subjects with a diagnosis of AD were placed in the AD group regardless of ODS scores (n=18 subjects). Measurements taken after moisturizer application are not included. The red line indicates median. The top and bottom edges of the box indicate 75th and 25th percentiles, respectively. The notch boundaries indicate 95% CI of the median. The upper and lower whisker bounds are defined as 75th percentile plus 1.5*IQR and 25th percentile minus 1.5*IQR, respectively. Red crosses indicate outliers. Pairwise comparison from post-hoc analysis are included (ns−p>0.05, * p<0.05, ** p<0.01, *** p≤0.001).

FIG. 30 shows comparison of measurements taken with the Delfin MoistureMeterD vs. SHS. Each data point represents the mean measurement from both devices at the same location under the same conditions (e.g. the left arm prior to moisturizer application). The blue line shows the regression and the shaded area shows 95% CI of the regression line. Both the SHS and Delfin MoistureMeterD measure in arbitrary units, but the scales differ. Measurements taken before and after moisturizer application in the study extension are included.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Visual diagnosis and tracking of skin diseases requires significant expertise and training. Existing diagnostic tools range from invasive procedures (e.g., skin biopsy) to imaging techniques (e.g., dermoscopy, confocal microscopy) which are expensive, infrequent, time-consuming, and require expertise to deploy. Noninvasive, point of care tools like corneometers have limited diagnostic value, interrogate only the top surface of the skin (about 15 μm), and are prone to measurement error due to high inter-rater variability, limiting use to research applications.

One of the objectives of this invention is to provide a soft, battery-free, flexible, non-invasive, reusable, low-cost, fully-integrated hydration sensor that is capable of monitoring and diagnosis of skin diseases in any environment.

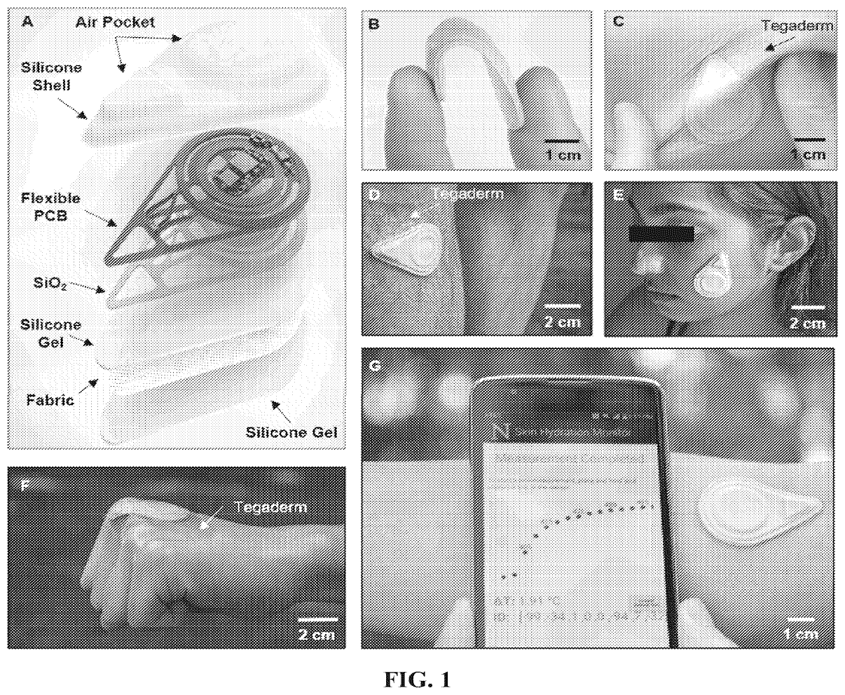

Referring to FIG. 1, the hydration sensor in one embodiment includes a top layer for thermal, chemical and mechanical isolation of the hydration sensor from the environment; a bottom layer operably placed on a target area of interest of a skin; and a flexible printed circuit board (f-PCB) disposed between the top layer and the bottom layer.

In one embodiment, the top layer is a shell-like top encapsulation layer including small air gaps/pockets (panel A of FIG. 1) for thermally insulating the critical sensing components. The top layer may comprise one or more air pockets/gaps/shells which thermally isolate the sensing area of the hydration sensor from the environment, thereby improving hydration sensor sensitivity; a shell made of silicone or similar materials, preferably hypoallergenic, to chemically and mechanically isolate and protect the underlying layers of the hydration sensor from external elements including water, dust, and/or from user touch; or a hollow shell-like structure to provide a soft touch or feel to the user.

In one embodiment, the top layer is formed of a flexible material including silicone or silicone gel, low/high density polyethylene (LDPE/HDPE), polystyrene, Teflon®, and various other flexible polymers. In one embodiment, the top layer is fabricated using molds to cure the silicone or similar materials, preferably hypoallergenic.

The bottom layer operably serves as a direct interface between the f-PCB and the skin. In one embodiment, the bottom layer comprises a flexible adhesive layer bonding to a thin layer of SiO₂ that is sputter-coated on a backside of the f-PCB. In one embodiment, the bottom layer adheres to the f-PCB through use of silicone bonding material, epoxy, glue, or commercial adhesive. The bottom layer may further comprise an ultrathin fabric of fiberglass or a reinforcement material embedded in the flexible adhesive layer for enhancing the mechanical robustness of the hydration sensor. In one embodiment, the reinforcement material is flexible and has varying mesh density and thickness to lend tear resistance to the bottom layer. In one embodiment, the flexible adhesive layer is formed of silicone or silicone gel, or commercially available double-sided skin-safe adhesives (preferably hypoallergenic), with the ratio of silicone and silicone gel being adjusted to co-optimize mechanical integrity and tackiness of the adhesive.

The f-PCB contains electronics for sensing and wireless communication. In one embodiment, the f-PCB is formed of a flexible material. In one embodiment, the flexible material comprises polyimide (PI) polyethylene terephthalate (PET), or any one of them in combination with a stiff PCB material including FR-4. In one embodiment, the f-PCB comprises open spaces and/or mechanical relief cuts for enhancing the overall flexibility, and limiting lateral thermal transport through the PI, away from the sensing components.

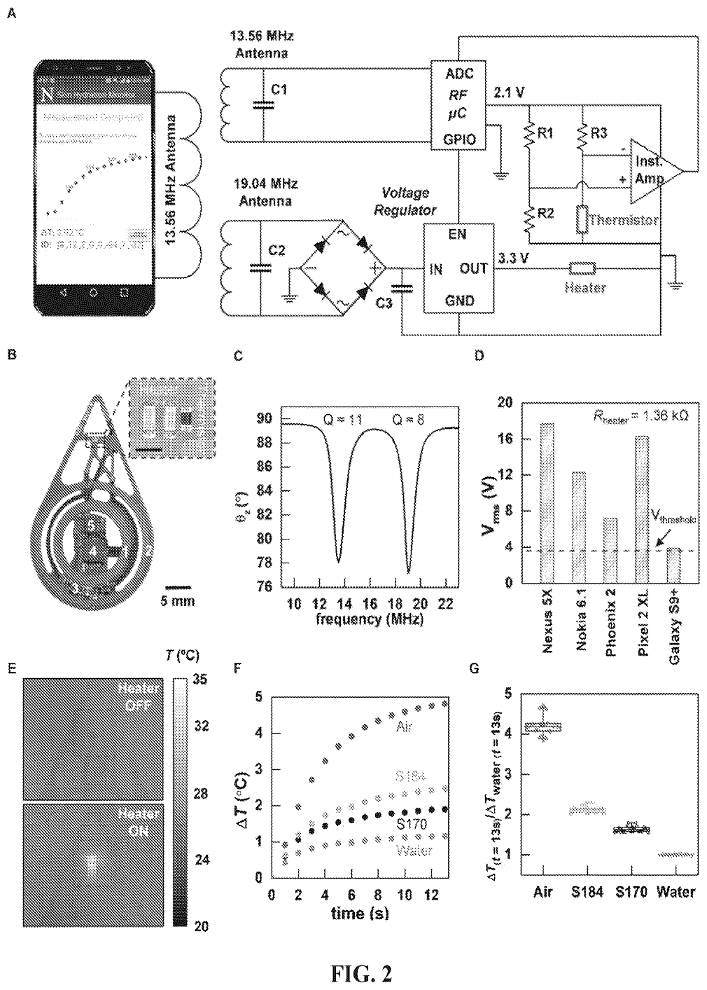

In one embodiment, the electronics, as shown in panels A-B of FIG. 2, comprises a heating circuit comprising a heating element for operably heating the target area of interest of the skin; and a sensing circuit comprising a temperature sensor for simultaneously recording a transient temperature change (ΔT) thereof. In one embodiment, the heating element and the temperature sensor are arranged laterally away from each other by a distance. The distance is determined by the design requirement of depth sensitivity into the skin, and ranges from 10s of μm to a few mm. In one embodiment, the heater and temperature sensor are the same component, where the distance between them is zero.

In one embodiment, the heating element comprises a heater comprising at least one resistor. The at least one resistor comprises two or more of surface-mount (SMT) thin film resistors, thick film resistors, through-hole resistors, and/or ultrathin-film (about 50-200 nm thick) metal resistors connected in series to form a heater. In one embodiment, the temperature sensor comprises an SMT negative temperature coefficient thermistor, positive temperature coefficient thermistor, resistance temperature detector (RTD), thermocouple or any other conductive temperature sensor.

In one embodiment, the temperature change (ΔT) in the temperature sensor is operably captured by an ADC of the microcontroller and transmitted to the external device.

In one embodiment, the sensing circuit further comprises a microcontroller (C), and the heater is operably switchable between ON and OFF controlled by the microcontroller. In one embodiment, the microcontroller is programmable with custom-designed embedded codes using at least one of NFC, Wi-Fi/Internet, Bluetooth, BLE, and Cellular communication protocols for wireless communication of the hydration sensor to a custom smartphone application.

In one embodiment, the electronics further comprises a primary antenna tuned to primary frequency, and a secondary antenna.

In one embodiment, the primary antenna is a transmission coil of an external device that is capable of wireless communications using the NFC protocol. In one embodiment, the external device is a smartphone, a tablet, a computer, or any electronic device with data reading capability.

In one embodiment, the secondary antenna comprises a first antenna electronically connected to the microcontroller for powering the sensing circuit and for communicating data to the external device using the NFC protocol, and a second antenna electronically connected to the heating circuit for powering the heating element. In one embodiment, the first antenna and the second antenna are arranged in a concentric geometry.

In one embodiment, the first antenna has a quality-factor (Q) that is relatively high to enable good communication distance and coupling across external devices with different primary antennae, and the second antenna has the quality-factor that is relatively low to support adequate power harvesting despite the difference between its resonance frequency and that of the primary coil. In one embodiment, the quality-factor of the first antenna is about 11, and the quality-factor of the second antenna is about 8.

In one embodiment, the first antenna is tuned to the primary frequency, and the second antenna is tuned to a secondary frequency that is different from the primary frequency so as to prevent interference with the first antenna. In one embodiment, the primary frequency is a standard NFC frequency of about 13.56 MHz, and the secondary frequency is about 19.04 MHz.

In one embodiment, the electronics comprises a generic design including blocks of (a) a powering system comprising voltage/power regulators driven by an external battery or magnetic induction to supply power to the heater, sensing and communication circuits; (b) an ADC (analog to digital convertor) chip or data modulator to prepare the output of the sensing circuit for transmission to an external readout device; and (c) a transceiver chip having at least one of at least one of NFC, Wi-Fi/Internet, Bluetooth, BLE, and Cellular communication protocols to transmit the output signal to the external readout device.

In one embodiment, the hydration sensor has a dynamic temperature range of about 18-45° C., adjustable through an amplifier gain, with a minimum resolution of about 15 mK, limited by the ADC.

In one embodiment, the hydration sensor has low flexural rigidity and effective modulus, to facilitate application even on highly curved features of the skin.

The hydration sensor is a soft, thin, wireless, and battery-free skin hydration sensor, and compatible for use in conjunction with other adhesives/tapes/bandages for example Tegaderm™, Scotch Tape® surgical tape, for applications on highly curved features on the human body/skin.

The thermal properties of the skin comprise thermal conductivity (k) and thermal diffusivity (α) of the skin that are related to water content (φ) of the skin. The water content is a function of a skin depth.

In one embodiment, an aggregate water content φ comprising the full measurement volume serves as a quantitative metric for diagnosis of skin diseases or of an efficacy of a treatment of a skin disease.

In one embodiment, the water content φ_{E} (epidermis) and φ_{D} (dermis), and skin surface temperature T₀ are determined from the measured temperature change ΔT vs. time t.

In one embodiment, the water content φ_{E} (epidermis) and φ_{D} (dermis), and skin surface temperature T₀ are used to determine a normal state or a disease state of the skin.

In one embodiment, the water content φ_{E} (epidermis) and φ_{D} (dermis), and skin surface temperature T₀ are used to diagnose various skin diseases.

In one embodiment, the water content φ_{E} (epidermis) and φ_{D} (dermis), and skin surface temperature T₀ are serve as quantitative metrics of an efficacy of a treatment of a skin disease.

The hydration sensor is usable for monitoring the skin condition, or other health and wellness products including skin moisturizers, lotions, and/or creams.

The hydration sensor is also usable for delivering treatment, monitoring the effects, modulating the treatment protocol as necessary, and/or potentially predicting for flares based on quantitative, individualized measurements on specific lesion sites.

The hydration sensor is further usable for monitoring water content of internal organs for various diseases where traditional monitoring techniques fail to offer continuous assessment of organ health. In one embodiment, the hydration sensor is usable for monitoring organs during organ transport for applications in organ transplant.

The hydration sensor can be used to monitor hydration status of healthy skin for applications in cosmetics, sports, and health/wellness. The skin health data (hydration of dermis, epidermis and skin temperature) for both lesions/healthy sites can be shared with physicians for remote medical care applications.

The hydration sensor is re-usable and removal without irritation to the skin or damage to the hydration sensor.

The hydration sensor is usable in both clinical and at-home settings.

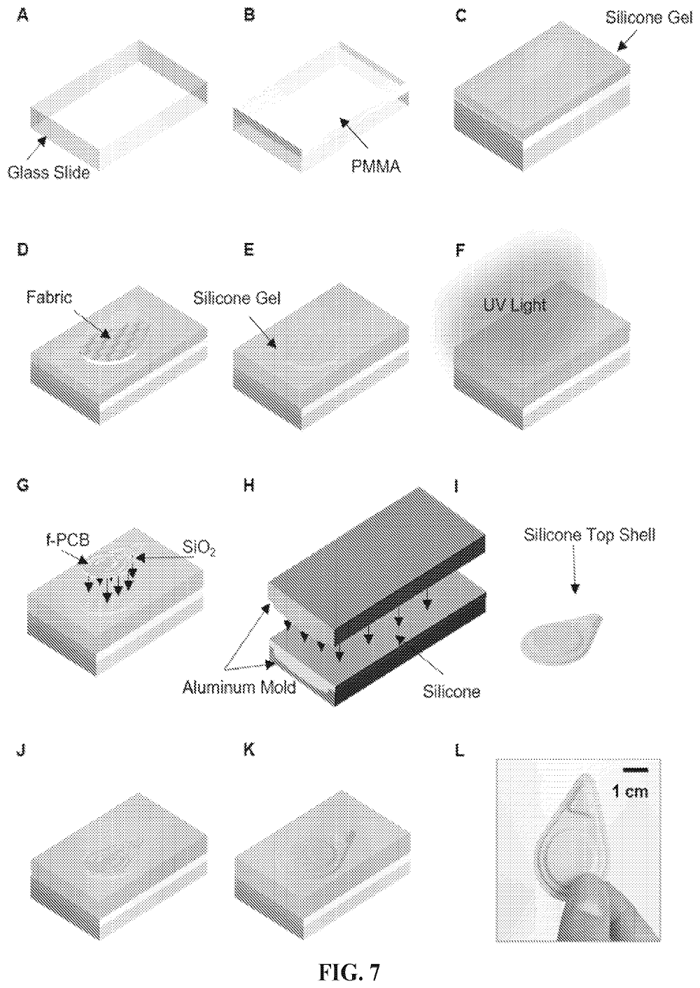

In another aspect, the invention relates to s method of fabricating a hydration sensor. Referring to FIG. 7, the method includes forming a bottom layer comprising a layered structure of a first flexible layer, a second flexible layer, and a fabric of fiberglass or a reinforcement material embedded between the first flexible layer and the second flexible layer (panel A of FIG. 1); treating a surface of the bottom layer with ultraviolet (UV) light to create reactive —OH groups on the surface of the bottom layer; sputter coating a layer of SiO₂ onto a backside of an f-PCB, wherein the f-PCB contains electronics for sensing and wireless communication; adhering the f-PCB to the bottom layer via a covalent dehydration reaction between the —OH groups on the layer of SiO₂ sputter coated onto the backside of the f-PCB and the treated surface of the bottom layer; forming a top shell layer including small air gaps; placing the top shell layer and the bottom layer such that the f-PCB is positioned between the top shell layer and the bottom layer and subsequently curing them on a hotplate so as to seal them; and cutting the sealed structure out in a desired shape to form the hydration sensor.

In one embodiment, each of the first flexible layer and the second flexible layer is formed of silicone or silicone gel, or commercially available double-sided skin-safe adhesives (preferably hypoallergenic), with the ratio of the silicone and silicone gel being adjusted to co-optimize mechanical integrity and tackiness of the adhesive. In one embodiment, the reinforcement material is flexible and has varying mesh density and thickness to lend tear resistance to the bottom layer.

In one embodiment, the bottom layer adheres to the f-PCB through use of silicone bonding material, epoxy, glue, or commercial adhesive.

In one embodiment, the top shell layer is formed of silicone or silicone gel, low/high density polyethylene (LDPE/HDPE), polystyrene, Teflon®, and various other flexible polymers.

In one embodiment, the top layer comprises an air pocket/gap/shell which thermally isolates the sensing area of the hydration sensor from the environment, thereby improving hydration sensor sensitivity; a shell made of silicone or similar materials, preferably hypoallergenic, to chemically and mechanically isolate and protect the underlying layers of the hydration sensor from external elements including water, dust, and/or from user touch; or a hollow shell-like structure to provide a soft touch/feel to the user.

In one embodiment, the top layer is fabricated using molds to cure the silicone or similar materials, preferably hypoallergenic.

In one embodiment, the f-PCB is formed of a flexible material comprising polyimide (PI), polyethylene terephthalate (PET), or any one of them in combination with stiff PCB material such as FR-4. In one embodiment, the f-PCB comprises open spaces and/or mechanical relief cuts for enhancing the overall flexibility, and limiting lateral thermal transport through the PI, away from the sensing components.

In one embodiment, the electronics comprises a heating circuit comprising a heating element for operably heating the target area of interest of the skin; and a sensing circuit comprising a temperature sensor for simultaneously recording a transient temperature change (ΔT) thereof. In one embodiment, the heating element and the temperature sensor are arranged laterally away from each other by a distance. In one embodiment, the distance is determined by the design requirement of depth sensitivity into the skin, and ranges from 10s of μm to a few mm. In one embodiment, the heater and temperature sensor are the same component, wherein the distance between them is zero.

In one embodiment, the sensing circuit further comprises a microcontroller (C). In one embodiment, the microcontroller is programmable with custom-designed embedded codes using at least one of NFC, Wi-Fi/Internet, Bluetooth, BLE, and Cellular communication protocols for wireless communication of the hydration sensor to a custom smartphone application.

In one embodiment, the electronics further comprises a primary antenna tuned to a primary frequency, and a secondary antenna.

In one embodiment, the primary antenna is a transmission coil of an external device that is capable of wireless communications using the NFC protocol. In one embodiment, the external device is a smartphone, a tablet, a computer, or any electronic device with data reading capability.

In one embodiment, the secondary antenna comprises a first antenna electronically connected to the microcontroller for powering the sensing circuit and for communicating data to the external device using the NFC protocol, and a second antenna electronically connected to the heating circuit for powering the heating element. In one embodiment, the first antenna and the second antenna are arranged in a concentric geometry.

In one embodiment, the first antenna is tuned to the primary frequency, and the second antenna is tuned to a secondary frequency that is different from the primary frequency so as to prevent interference with the first antenna.

In one embodiment, the hydration sensor can be operated using a single antenna.

In one embodiment, the electronics comprises a generic design including blocks of (a) a powering system comprising voltage/power regulators driven by an external battery or magnetic induction to supply power to the heater, sensing and communication circuits; (b) an ADC (analog to digital convertor) chip or data modulator to prepare the output of the sensing circuit for transmission to an external readout device; and (c) a transceiver chip having at least one of NFC, Wi-Fi/Internet, Bluetooth, BLE, and Cellular communication protocols to transmit the output signal to the external readout device.

In yet another aspect, the invention relates to a method d of monitoring and/or diagnosing a condition of a skin. The method includes attaching a hydration sensor onto a target area of interest of the skin, wherein the hydration sensor comprising an f-PCB containing electronics for sensing and wireless communication, wherein the electronics comprising a heating circuit comprising a heating element; a sensing circuit comprising a temperature sensor; a microcontroller (μC) coupled to the heating element and the sensing circuit, and a transceiver coupled to the heating element, the sensing circuit and the microcontroller; heating the target area of interest of the skin by the heating element and simultaneously recording a transient temperature change (ΔT) thereof by the temperature sensor; obtaining water content of the target area of interest of the skin from the temperature change (ΔT); and determining a condition of the skin at the target area of interest based on the obtained water content. In one embodiment, the water content comprises water content φ_{E} of the epidermis and water content φ_{D} of the dermis.

In one embodiment, the step of obtaining the water content comprises separately determination of φ_{E} and φ_{D} from the temperature change ΔT vs. t curves, thereby enabling real-time display of φ_{E} and φ_{D} on the phone application shortly after completing the measurement.

In one embodiment, the transceiver transmits data through a wireless communication protocol including at least one of NFC, Wi-Fi/Internet, Bluetooth, BLE, and Cellular communication. In one embodiment, the transceiver relays data to an external device through various wireless communication methods including at least one of NFC, Wi-Fi/Internet, Bluetooth, BLE, and Cellular communication protocols.

In one embodiment, the transceiver is powered wirelessly or through use of a battery/capacitive discharge mechanism.

In one embodiment, the transceiver comprises a primary antenna tuned to primary frequency, and a secondary antenna. In one embodiment, the primary antenna is a transmission coil of an external device that is capable of wireless communications using the NFC protocol. In one embodiment, the secondary antenna comprises a first antenna electronically connected to the microcontroller for powering the sensing circuit and for communicating data to the external device using the NFC protocol, and a second antenna electronically connected to the heating circuit for powering the heating element. In one embodiment, the first antenna and the second antenna are arranged in a concentric geometry. In one embodiment, the first antenna is tuned to the primary frequency, and the second antenna is tuned to a secondary frequency that is different from the primary frequency so as to prevent interference with the first antenna.

In one embodiment, the method further comprises programming the microcontroller with a custom-designed embedded code using an NFC read/write interface allowed for wireless communication of the hydration sensor to a custom smartphone application.

In one embodiment, the step of heating the target area of interest of the skin comprises powering the heating element through a regulated DC supply at a voltage of about 3.3 V derived from the secondary AC voltage at the second antenna, resulting in a constant thermal power of q=10 mW/mm².

In one embodiment, the temperature sensor connects as one of the arms of a Wheatstone bridge powered by a rectified 2.1 V from the RF μC, wherein the voltage across the arms of the Wheatstone bridge is amplified, subsequently read by the RF μC ADC, then transmitted through the first antenna to the phone NFC reader, and recorded in the phone's memory, and displayed on the screen.

In one embodiment, the step of determining the condition of the skin at the target area of interest comprises comparing the obtained water content to a standard water content at the target area of interest so as to determine a normal state or a disease state of the skin.

In one embodiment, the step of determining the condition of the skin at the target area of interest comprises diagnosing a skin disease at the target area of interest based on wherein the obtained water content thereof.

In one embodiment, the step of determining the condition of the skin at the target area of interest comprises diagnosing a skin disease at the target area of interest by comparing measurements of skin barrier function to a clinician-derived overall dry skin (ODS) score.

In one embodiment, the step of determining the condition of the skin at the target area of interest comprises evaluating an efficacy of a treatment of the skin disease.

In one embodiment, the method further comprises one or more steps of delivering treatment, monitoring the effects, modulating the treatment protocol as necessary, and/or potentially predicting for flares based on quantitative, individualized measurements on specific lesion sites.

In one exemplary embodiment, a soft, battery-free, flexible, non-invasive, reusable skin hydration sensor (SHS) adherable to even small-areas and curvilinear surfaces of the body is disclosed. The sensor platform/system operably measures the volumetric water content in skin as a function of depth (up to about 1 mm), and wirelessly transmits data to any near field communication (NFC) compatible smartphone. In certain embodiments, the hydration sensor system comprises off-the-shelf electronic components in a manufacturable layout, along with unique powering strategies, and encapsulation procedures, leading to robust device operation with high precision (±5%) measurements of water content and high-resolution (±0.015° C.) assessments of skin surface temperature. Validation on n=16 healthy/normal human subjects reveals an average water content of about 63% in the skin across multiple body locations. Pilot studies on patients with skin diseases, including atopic dermatitis (AD), psoriasis, urticaria, xerosis cutis, and rosacea, illustrate the capability of the hydration sensor to categorize skin diseases and aid in future diagnosis (φ_{AD}=0.0034). A demonstration includes monitoring changes in hydration level for a diseased subject with the application of topical treatments.

The hydration sensor has potential to be made very low cost and can be made ubiquitous because it pairs easily with smart devices. Measurements made by the hydration sensor can be recorded in an application that will keep track of skin hydration measurements and to help relay data with a physician if required. The device is prime for FDA approval for several dermatological diseases as it is noninvasive, very low-risk, and made of bio-compatible materials which interface with the skin.

Among other things, the hydration sensor can be used for hydration measurements of skin for cosmetics applications, including, but are not limited to, hydration measurements of skin to assess topical creams/ointments/moisturizers; hydration measurements of skin to determine severity of dermatological diseases; hydration measurements of skin to aid in dermatological disease diagnosis; hydration measurements on plants to determine water uptake and help determine optimal irrigation strategies for applications in agriculture; hydration measurements of internal organs to monitor organ health.

In addition, the hydration sensor can be used for applications to measure thermal conductivity, thermal diffusivity, heat capacity and other thermal properties of any material as a function of depth.

The hydration sensor can also be used for applications to measure water content of any material surface as a function of depth, including hydrogels, plants (irrigation and agriculture applications), food preservation (dried food products, grains, fruits, meats), and/or concrete (industrial applications).

The hydration sensor can be used to monitor composition of food/beverages, medicines/industrial chemicals.

The hydration sensor is compatible with alcohol-based cleaning wipes allowing for re-use across different users, without any damage to the hydration sensor or loss in efficacy of the hydration sensor adhesive.

The hydration sensor can be sterilized using alcohol, autoclave steam sterilization, and gas phase sterilization.

The hydration sensor can easily be translated to other wireless powering/data transfer technologies, and allows non-invasive measurements of skin hydration beyond the most superficial layers of the skin.

The hydration sensor platform has at least the following advantages:
Extracts percent by volume water content in skin as opposed to arbitrary units of measure (e.g., arbitrary corneometer units).
Pairs with smart devices (phone, laptop, tablet, etc.) for powering/data transfer, allowing ease of use.
Comprised of low-cost materials compared to price of commercial corneometers on the market.
Comprised of off-the-shelf electronic components and compatible with commercial manufacturing techniques.
Able to harvest wireless power/perform wireless charging through separate, de-tuned radio frequency (RF) coil to enable high-power battery-less measurements.
Makes use of a custom fiberglass-reinforced silicone gel adhesive as an interface between the skin and the device, enabling tear-resistance and mechanical robustness.
Uses two surface mount resistors spaced a very small distance away from a negative temperature coefficient thermistor as a separated heater/sensor structure to perform transient plane source measurements.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Reliable, Low-Cost, Fully Integrated Hydration Sensors for Monitoring and Diagnosis of Inflammatory Skin Diseases in any Environment

Visual diagnosis and tracking of skin diseases requires significant expertise and training. Existing diagnostic tools range from invasive procedures (e.g., skin biopsy) to imaging techniques (e.g., dermoscopy, confocal microscopy) which are expensive, infrequent, time-consuming, and require expertise to deploy. Noninvasive, point of care tools like corneometers have limited diagnostic value, interrogate only the top surface of the skin (˜15 μm), and are prone to measurement error due to high inter-rater variability, limiting use to research applications.

This exemplary study reports a set of results in materials, device designs and analysis approaches that address all these aforementioned challenges, in the form of a soft, thin, wireless, and battery-free skin hydration sensor (SHS) and corresponding models that can be used to accurately assess water content of skin regardless of body location or environment. Careful systems-level engineering enables highly robust, reliable measurements and with platforms that are compatible with established manufacturing techniques for consumer electronics gadgetry, with readily available off-the-shelf components. Theoretical modeling establishes a means to directly determine the volumetric water content in skin as a function of depth, from the raw data obtained using the sensors. Benchtop characterization tests and studies using healthy/normal subjects establish the accuracy and reliability of the SHS devices, as well as the key engineering parameters that affect these quantities. Key results include clinical use of the SHS on n=13 patients with a wide range of inflammatory skin conditions (e.g., atopic dermatitis, psoriasis, urticaria, xerosis cutis, and rosacea), with benchmarks against standard tools, to quantitatively characterize the diseased locations. Additional clinical demonstrations include tracking improvements in skin water content after application of a topical moisturizer.

The objective of this exemplary study is to design and demonstrate a robust, wireless, battery-free and soft sensor for widespread, non-invasive monitoring of skin hydration independent of clinical settings, and to validate their performance against clinical standard tools. Patients recruited received full informed consent (Ann & Robert H. Lurie Children's Hospital of Chicago, Chicago, IL; IRB Study #STU00209010). Inclusion criteria specified healthy/normal subjects or patients undergoing evaluation or routine-checkups for clinical skin-related pathologies. A hypoallergenic silicone-based surgical tape (Kind Removal Silicone Tape, 3M, Inc., USA) assisted in adhering the device to the skin. Sterilization of the moisture meters and SHS required individual wiping down of all surfaces with single-use alcohol wipes (Sterile Alcohol Prep Pads, Dynarex Corp., USA).

Patients with Disease: The sensor recorded measurements in triplicate on identified lesions, perilesional locations, or unaffected contralateral locations if no perilesional area with a normal appearance was available. A waiting period of 30 s after application of the device to skin and between measurements ensured the sensing components achieved thermal equilibrium with the temperature of the skin. Corneometer readings (gpskin Barrier, GPower, Inc., KR) taken in triplicate on the same corresponding sites served as a metric for comparison to the measurements taken by the SHS.

Patients with healthy normal skin: Measurements using the SHS device were only performed once on each body location to imitate a real-life use-case. Body locations selected for studies on healthy/normal subjects included the forehead, cheek, volar forearm, shin, calf, and heel. Measurements were not performed in cases with presence of excessive hair at the measurement site or for those who had applied moisturizer to that region of skin. A hypoallergenic silicone-based surgical tape (Kind Removal Silicone Tape, 3M, Inc., USA) assisted in adhering the device to the skin. Sterilization of the moisture meters and SHS required individual wiping down all surfaces with single-use alcohol wipes (Sterile Alcohol Prep Pads, Dynarex Corp., USA).

### Fabrication of Skin Hydration Sensors

Initial prototypes and proof-of-concept devices involved use of a laser cutter (LPKF U4, LPKF Inc., DE) to pattern a double-sided copper-clad laminate (Pyralux AP8535R, DuPont, Inc., USA) and standard micro-soldering techniques. To avoid damage to the critical sensing components, soldering the thermistor (NTCG063JF103FT, TDK Corporation, Japan) and heater resistors (RR0306P-681-D, Susumu Co., Ltd., Japan) using a low-temperature solder paste (TS391AX10, Chip Quik Inc., USA) and heat gun temperature of 200° C. for less than 5 seconds was the final step in component assembly. Initial devices were prototyped in a lab setting, and the final designs were then sent to an external ISO-9001 compliant vendor for full manufacturing and assembly of multiple f-PCBs to illustrate the compatibility with readily available outsource manufacturing techniques. Programming the microcontroller (RF430FRL152H, Texas Instruments, Inc., USA) with a custom-designed embedded code using an NFC read/write interface (TRF7970AEVM, Texas Instruments, Inc., USA) allowed for wireless communication of the sensor to a custom smartphone application. This software application features tunable measurement time and custom file naming.

### Encapsulation of Skin Hydration Sensors

Briefly, preparing a layered structure of about 45 μm thickness of silicone (Ecoflex 00-30, Smooth-On, Inc., USA)/silicone gel (Ecoflex gel, Smooth-On, Inc., USA), about 30 μm thin fiberglass fabric, followed by an additional about 45 μm thin silicone/silicone gel on a glass slide yielded the bottom layer of the SHS device, as the direct interface between the f-PCB and the skin. The f-PCB adhered to the silicone layer via bonding between a thin coating of SiO₂ sputter deposited onto the backside of the f-PCB and dangling —OH bonds on the surface of the silicone (formed through UV-light functionalization). A custom-made aluminum mold and a hot press (Carver Press, Carver Inc., USA) allowed formation of a structured film of silicone (about 2.7 mm thickness) as the top shell of the device. Screen printing uncured silicone beyond the outermost border of the f-PCB, placing the top shell and bottom layer together and subsequently curing of the entire device on a hotplate at about 70° C. for about 10 min sealed the system.

Cutting the completed structure out in a teardrop shape using a die cutter and peeling the structure off the glass slide completed the fabrication process.

The detailed fabrication procedures in certain embodiments are shown in FIG. 7 and described as follows:
1. Clean a glass slide with acetone, isopropyl alcohol, and blow dry with nitrogen (step A of FIG. 7).
2. Spin coat a layer of poly (methyl) methacrylate (495 PMMA A5, MicroChem Corp., USA) on the glass slide at about 3000 rpm, and bake at about 180° C. for about 3 minutes (step B of FIG. 7).
3. Pour about 75% by weight of Ecoflex gel (Smooth-On, Inc., USA) and about 25% Ecoflex 00-30 (Smooth-On, Inc., USA) into a container. Then add about 2% by weight of light blue dye (Silc Pig, Smooth On, Inc, USA) to the container. Centrifuge at about 2000 rpm for about 30 s.
4. Spin coat a layer of this silicone mixture onto the PMMA coated glass slide at about 3000 rpm, and then let cure on a hotplate at about 70° C. for about 5 minutes (step C of FIG. 7).
5. Place a cut piece of fiberglass on top of the cured PDMS and apply light pressure to ensure robust Van der Waals adhesion (step D of FIG. 7).
6. Spin coat an additional layer of the silicone mixture on top of the fiberglass at about 3000 rpm and let cure on a hotplate at about 70° C. for 5 minutes (step E of FIG. 7).
7. Treat the surface of the bottom layer silicone with ultraviolet (UV) light to create reactive —OH groups on the surface of the silicone (step F of FIG. 7).
8. Sputter SiO₂ onto the back of the flexible printed circuit board (f-PCB) and place onto the cured silicone slide to allow a covalent dehydration reaction between the —OH groups on the surface of the silicone and the SiO₂ (step G of FIG. 7).
9. Clean both halves of the aluminum top shell mold with acetone, isopropyl alcohol, and blow dry with nitrogen (step H of FIG. 7).
10. Pour Ecoflex 00-30 into a container and add about 2% by weight of light blue dye. Centrifuge at about 2000 rpm for about 30 s.
11. Pour Ecoflex 00-30 into the concave mold and put both parts of the mold together. Cure in hot press (Carver Press, Carver Inc., USA) at about 250° C. and about 1000 lbs for about 1.5 minutes.
12. Cut out the cured top shell to the proper shape using the die cutter (step I of FIG. 7).
13. Apply Ecoflex 00-30 to the edges of the f-PCB and place the top shell on top (steps J-K of FIG. 7). Let cure on a hotplate at about 70° C. for about 10 minutes.
14. Cut out the final sensor using the die cutter and peel off the glass slide (step L of FIG. 7).

### Data Collection and Analysis

Powering the heater occurs through a regulated DC supply at a voltage of about 3.3 V derived from the secondary AC voltage at Ant. 2, resulting in a constant thermal power of q=10 mW/mm². The resistance of the heating element remains nearly constant due to its low temperature coefficient of resistance=25 ppm/° C. The thermistor connects as one of the arms of a Wheatstone bridge powered by a rectified 2.1 V from the RF μC. The voltage across the arms of the Wheatstone bridge is amplified, subsequently read by the RF μC ADC (sampling rate=1 Hz, resolution=10 bits), then transmitted to the phone (data rate=0.125 Hz) NFC ISO15693 reader, and finally recorded in the phone's memory, and also displayed on the screen. The application supports to the use of any smart devices such as Android phones with NFC capability.

The relationship between the thermistor resistance R and temperature T derived from the vendor datasheet is:

$$R = - ( \frac{1}{1.52 \times 1 {0}^{- 4}} ) log ( \frac{T - 7.42433}{79.54344} ) .$$

Referring to the circuit schematic shown in panel A of FIG. 2, the output voltage for the instrumentation amplifier is:

$${V}_{out} = g \times {V}_{D D} \times ( \frac{R 2}{R 1 + R 2} - \frac{R}{R 3 + R} ) ,$$

where R1=R2=10 kΩ, R3=10.7 kΩ, and V_{DD}=2.1 V. and g is the amplifier gain given by

$$g = 1 + \frac{100 k Ω}{{R}_{g}} ,$$

R_{g}=45.3 kΩ (gain resistor). Vₒᵤₜ is converted to a 10-bit digital output by the μC ADC which has an input range of 0-0.9V. Thus,

$$A D {C}_{bits} = {V}_{out} * \frac{{2}^{10} - 1}{0.9 V} ,$$

### Finite Element Analysis (FEA)

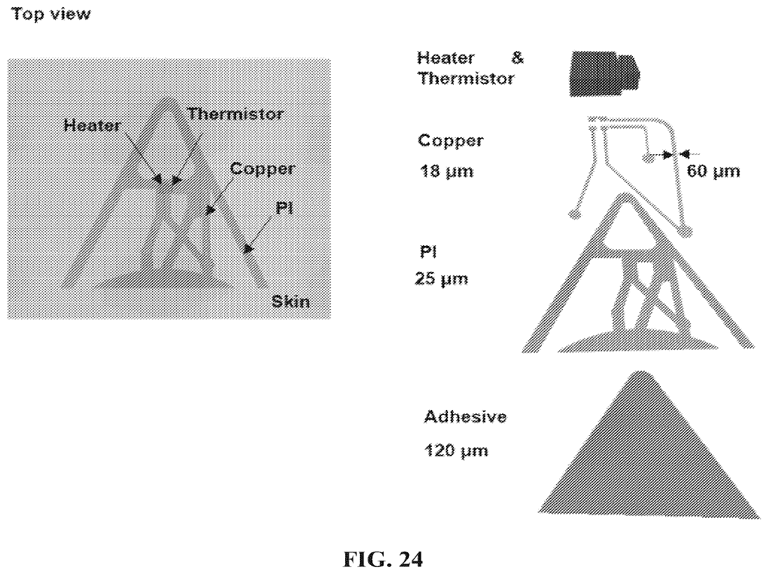

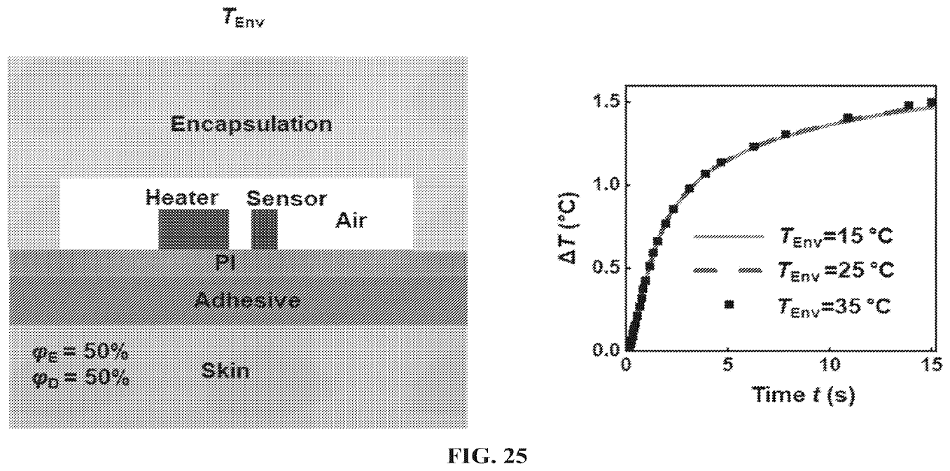

FEA was performed using the commercial software ABAQUS. As shown in FIG. 24, the model includes all parts of the device related to the transfer including heater, thermistor, copper wires, Polyimide (PI) substrate, Ecoflex adhesive, and measurement sample (epidermis, and dermis). The air gap on top of the device is modeled by the adiabatic boundary condition at the top surface. A refined mesh much smaller than the finest feature size of the device (about 18 μm, copper thickness) was adopted to guarantee simulation convergence and accuracy. The material parameters used in all simulations are k_{copper}=377 W/(m-K), α_{copper}=10⁹ mm²/s, k_{PI}=0.12 W/(m-K), α_{PI}=0.078 mm²/s, k_{Ecoflex}=0.21 W/(m-K), α_{Ecoflex}=0.091 mm²/s, with k and a standing for thermal conductivity and thermal diffusivity, respectively. FEA simulations on the influence of environmental temperature/humidity on the device are shown in FIG. 25.

### Statistical Analysis

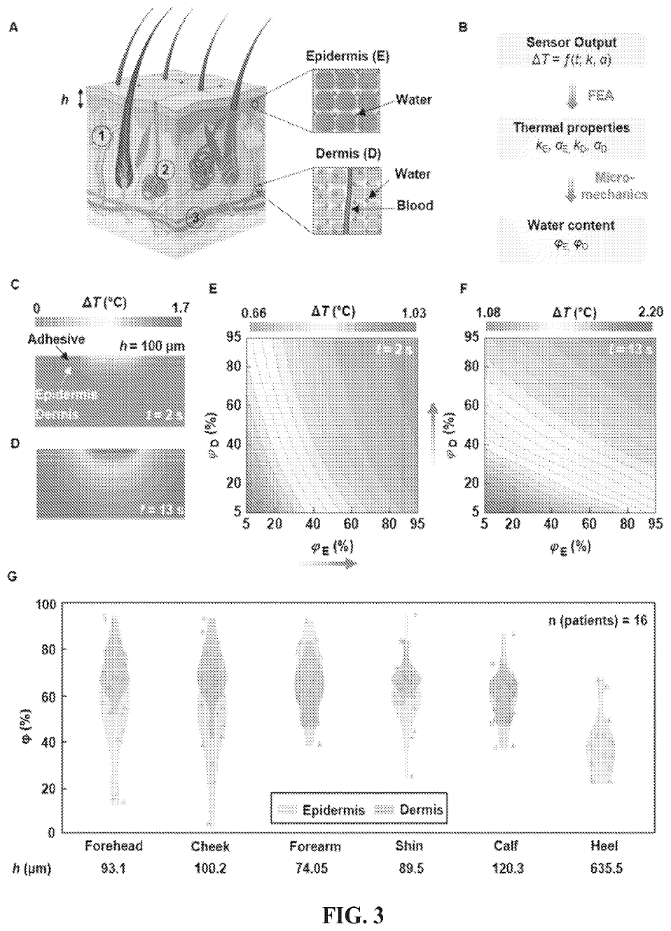

All P-values correspond to a two-sided Wilcoxon signed rank test with the null hypothesis H₀=N−L=0. Error bars in various data plots indicate the standard deviations over three consecutive trials on the substrate/skin. Subjects with dense coverage of hair and those who applied moisturizer were omitted from the data on healthy/normal subjects in panel F of FIG. 3.

### Device Structure and Operation

The SHS measures the thermal properties (conductivity k and diffusivity α) of the skin using the transient plane source (TPS) technique. TPS exploits Joule heating via a resistive element placed on the top of a sample of interest while simultaneously recording the transient temperature change (ΔT) of the heating element itself or of a separate temperature sensor. An exploded-view diagram of the SHS appears in panel A of FIG. 1. The flexible printed circuit board (f-PCB) contains electronics for sensing and wireless communication. Open spaces/mechanical relief cuts in the polyimide (PI) layer of the f-PCB enhance the overall flexibility, and limit lateral thermal transport through the PI, away from the sensing components. To ensure low thermal resistance to the skin, a thin bottom silicone gel adhesive (about 120 μm thick) bonds by a covalent dehydration reaction between —OH groups on the surface of the silicone to a thin layer of SiO₂ (about 75 nm) sputter-coated on the backside of the f-PCB. An ultrathin fabric of fiberglass (about 30 μm) embedded in the silicone gel greatly enhances the mechanical robustness of the system. Repeated application and removal of the device is possible without irritation to the skin or damage to the device. Cleaning with alcohol wipes provides a convenient means to sterilize the device for use across different patients with minimal risk and restores the tackiness of the adhesive by eliminating particulates or other contaminants (sensors were tested for at least 130× on average without damage, demonstrating their robustness). The shell-like top encapsulation layer creates small air gaps for thermally insulating the critical sensing components. This design also leads to low flexural rigidity and effective modulus, to facilitate application even on highly curved features of the body. Detailed fabrication procedures are described above and shown in FIG. 7.

The resulting hydration sensors are flexible, thin (about 2.1 mm), lightweight (about 2.5 g), and have small lateral dimensions (about 4.5 cm×3.1 cm) (panels B-C of FIG. 1), enabling gentle yet intimate contact with the surface of the skin for application on nearly any region of the body, including challenging areas such as the shin, face, and even the knuckles (panels D-F of FIG. 1). The SHS wirelessly receives power from and communicates with any standard smartphone that has near-field communication (NFC) capabilities (panel G of FIG. 1), thereby eliminating the need for a battery and allowing for a compact form factor. This collection of features suggests the potential for widespread use, not only in clinical and laboratory settings but also in the home or office. An easy-to-use, custom software application runs on the phone as an interface to allow patients to monitor their skin health and share data with their physician.

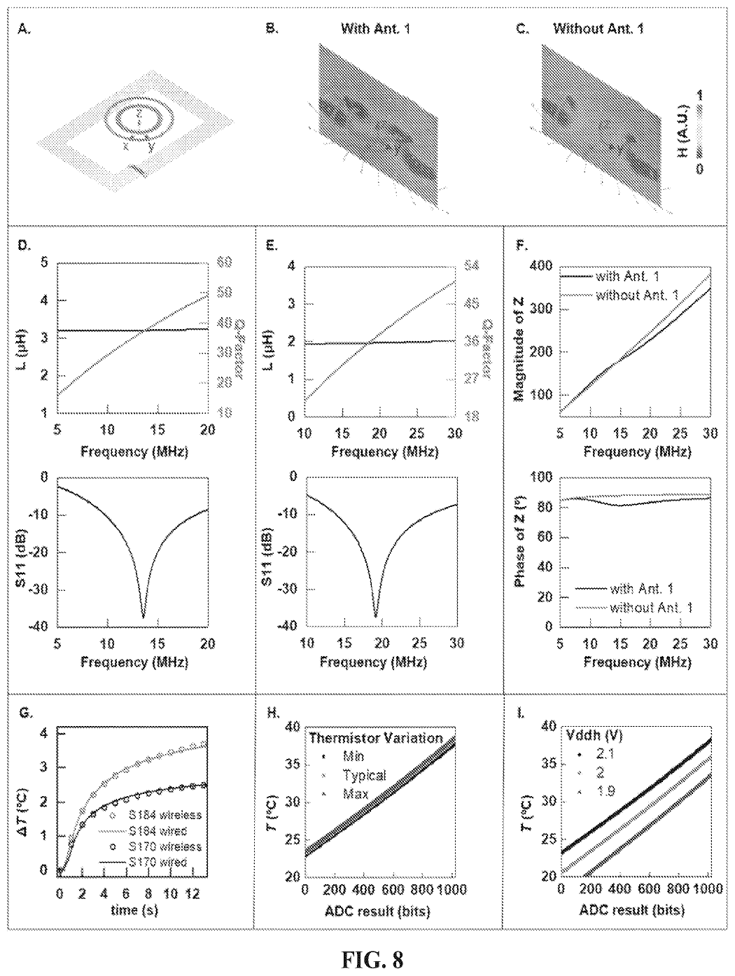

A detailed circuit diagram for the f-PCB electronics appears in panel A of FIG. 2. Accurate measurements require relatively high levels of heating power (about 10-60 mW). The transmission coil of the smartphone serves as the primary 13.56 MHz (standard NFC frequency) antenna. In certain embodiments, the sensor design involves separate power harvesting coil antennas for the heating and temperature sensing circuits, in a concentric geometry (panels A-C of FIG. 2). The secondary antenna 1 (Ant. 1), tuned to 13.56 MHz, connects to the RF microcontroller (μC) for powering the sensing circuit and for communicating data to the phone using NFC protocols. The secondary antenna 2 (Ant. 2), tuned to 19.04 MHz to prevent interference with Ant. 1, powers the heater (panel A of FIG. 2). The quality-factor (Q) of Ant. 1 is relatively high (about 11) to enable good communication distance and coupling across phones with different primary antennae, while the quality-factor for Ant. 2 is relatively low (about 8) to support adequate power harvesting despite the difference between its resonance frequency and that of the primary coil, as shown in panel C of FIG. 2. Detailed antenna characteristics and computational results on the interference between the two antennas are shown in FIG. 8. Rectification and subsequent voltage regulation of the harvested voltage from Ant. 2 produces a constant 3.3 V voltage source for the heater. For a wide variety of smartphones, the root-mean-square (RMS) voltage harvested from Ant. 2 (Vᵣₘₛ) is greater than the threshold voltage (V_{threshold}) required for stable voltage regulator operation (panel D of FIG. 2). Thus, the dual-coil powering scheme supports compatibility across a wide variety of smartphones.

The sensing component comprises two commercial surface-mount (SMT) thin film resistors (R=680±3.4Ω each) connected in series to form a heater (about 0.9×0.9 mm²) and an SMT negative temperature coefficient thermistor, placed 60 μm laterally away from the heater (panel B of FIG. 2, inset). The heater can be switched ON and OFF (see infrared camera image, panel E of FIG. 2) by the μC. The change in temperature of the thermistor is captured by the ADC of the μC and transmitted to the phone. A Bill of Materials (BOM) for all electronic components is listed in Table 1. The dynamic temperature range of the device is about 23-38° C., adjustable through the amplifier gain, with a minimum resolution of about 15 mK, limited by the ADC. Control experiments that use a wired system for power and data acquisition yield data that are indistinguishable from those captured using the NFC wireless system introduced here (FIG. 8). As further validation, the SHS output for ΔT vs. time (t) on standard materials with known k produces the expected, qualitatively inverse relationship between ΔT and k (panel F of FIG. 2). SHS measurements are calibrated using the ΔT vs. t curve for water (which has known k=0.6 W-K, α=0.14 mm²/s). The results of 5× measurements each performed using n=6 different sensors with the same standard materials as shown in panel F of FIG. 2 appear in normalized form at t=13 s in panel G of FIG. 2.

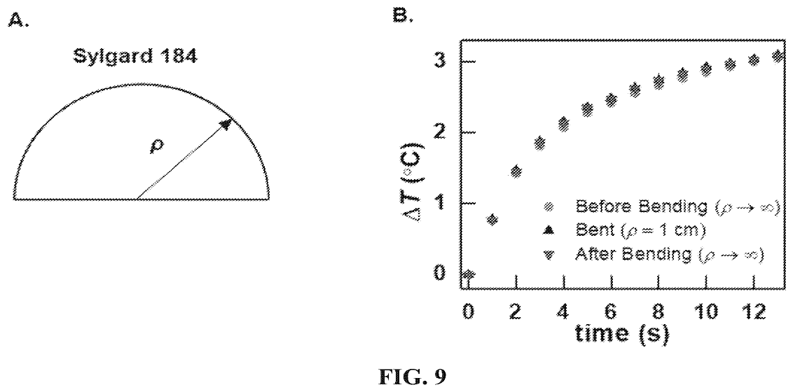

Measurements of ΔT for a single device are highly repeatable, with an error of only 2% (possible sources of variability are in FIG. 8). Measurements of ΔT on curved substrates confirm that the device remains largely unaffected by mechanical deformation (FIG. 9).

TABLE 1
Bill of Materials for all electronic components.

| | Quantity | | |
|---|---|---|---|
| | per | | |
| Component | sensor | Description | Manufacture part number |
| C1 | 2 | CAP CER 10000PF | GRM033R71A103KAO1D |
| | | lOy X7R 0201 | |
| C2 | 5 | CAP CER 0.1UF | GRM033C71C104KE14D |
| | | 16V X7S 0201 | |
| C3 | 2 | CAP CER 2.2UF | GRMs033R61A225ME47D |
| | | 10V X5R 0201 | |
| C4 | 1 | CAP CER 5.1PF sov | GRM0335C1H5R1BAO1D |
| | | coG/Npo 0201 | |
| C5 | 1 | CAP CER 4.7UF | GRM035R60.1475ME15D |
| | | 6.3V X5R 0201 | |
| C6 | 1 | CAP CER 1UF 10V | GRM033R61A105ME15D |
| | | X5R 0201 | |
| C7 | 1 | CAP CER 10UF | GRM188R61E106MA73D |
| | | 25V X5R 0603 | |
| C8 | 1 | CAP CER 27PF 50V | GRM0335C1H270.1A01D |
| | | COG/NPO 0201 | |
| R1 | 2 | RES SMD 0 OHM | ER. 1-1GNOR00C |
| | | JUMPER 1/20W | |
| | | 0201 | |
| R2 | 3 | RES 10K OHM 1% | RMCF0201FT10K0 |
| | | 1/20ni 0201 | |
| R3 | 1 | RES SMD 10.7K | CRCW020110K7FKED |
| | | OHM 1% | |
| | | 1/20Ar 0201 | |
| R4 | 1 | RES SMD 45.3K | RCO201FR-0745K3L |
| | | OHM 1% | |
| | | 1/20W 0201 | |
| R5 | 2 | RES SMD 680 OHM | RR0306P-681-D |
| | | 0.5% 1/20W 0201 | |
| Thermistor | 1 | THERMISTOR NTC | NTCG063.1F103FTB |
| | | 1OKOHM 3380K | |
| | | 0201 | |
| Schottky | 4 | DIODE SCHOTTKY | CDBQR023OR |
| Diode | | 30V 200MA 0402 | |
| RF μC | 1 | IC RFID TRANSP | RF430FRL152HCRGER |
| | | 13.56MHZ 24VQFN | |
| Inst. Amp. | 1 | IC INST AMP 1 | INA333AIDRGR |
| | | CIRCUIT 8SON | |
| Voltage | 1 | IC REG LINEAR | TPS70933DRVR |
| Regulator | | 3.3V 150MA 6SON | |

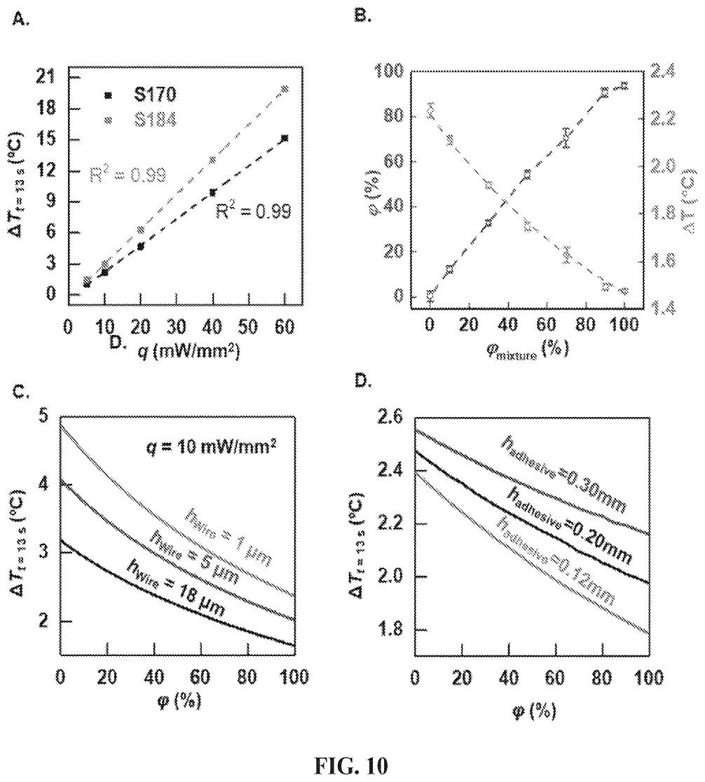

The sensitivity of the SHS (i.e., the range ΔT for materials across the span of physiological values of k) depends partly upon the fraction of the generated thermal power that passes through the sample of interest. Ideally, the thermal properties of the sample will dominate the response, ΔT. Alternate paths of thermal transport/thermal sinking, namely those that can occur within the device itself, reduce the sensitivity to the sample. Associated techniques to improve sensitivity include reducing the widths and thicknesses of interconnect metal (Cu) traces in the vicinity of the heater/thermistor, reducing the thickness of the adhesive, and increasing the thermal power density (FIG. 10).

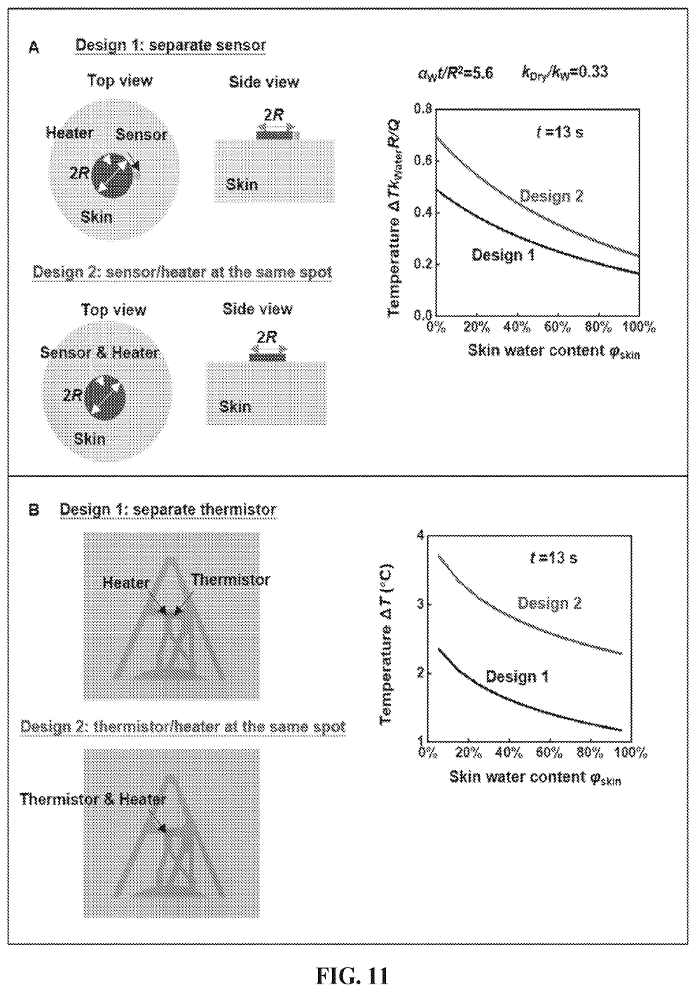

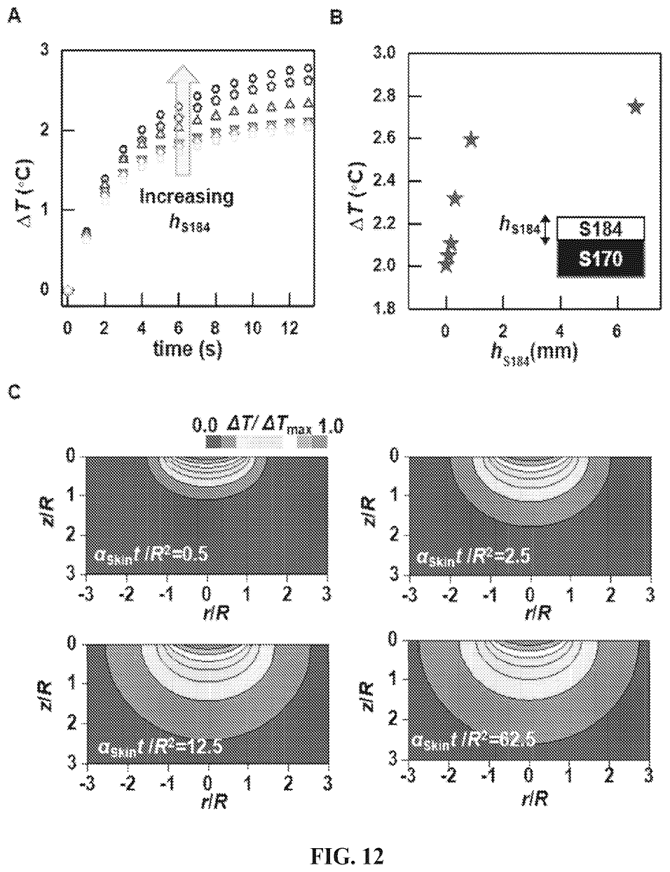

Sensor geometry can also alter measurement sensitivity, however due to the small (about 60 μm) spacing between the heater and thermistor, there is negligible difference in sensitivity between the case of a single component serving as both the heater and the thermistor and the separated heater/thermistor structure (FIG. 11). The distance between the heater and thermistor, as well as the total measurement time, determine the maximum measurement depth, which for the configuration reported here is about 1 mm (See FIG. 12 for details). Increasing the heater size and measurement time beyond the maximum of 13 s as is used in this work increases the measurement depth (about 6 mm depth experimentally achieved).

To predict the effect of some key design parameters, a simplified model is established herein. As shown in panel A of FIG. 11, the model includes a disk-shaped heater (radius R, heating power q per unit area, total heating power Q=πR²q negligible thickness) on a semi-infinite, homogenous skin (thermal conductivity kₛₖᵢₙ and thermal diffusivity αₛₖᵢₙ). A sensor with negligible size is placed right beside the heater (at r=R in the polar coordinate system). The temperature change in the sensor is

$$Δ {T}_{sensor} = \frac{q R}{{k}_{skin}} {\int }_{0}^{+ ∞} [ {J}_{0} ( x ) {J}_{1} ( x ) erfc ( - x \sqrt{\frac{t {α}_{s k i n}}{{R}^{2}}} ) ] \frac{d x}{x} ,$$

where J₀(x) and J₁(x) are the Bessel functions of the first kind with zero- and first-orders, respectively, and erfc(x) is the complementary error function. Combined with the micromechanics model Eqs. (1) and (2) below, ΔTₛₑₙₛₒᵣ can be related to the skin water content φₛₖᵢₙ. In some previous works, the heater itself serves as the sensor such as that the average temperature in heater is the sensor temperature, which is

$$Δ {T}_{h e a t e r} = \frac{2 q R}{{k}_{s k i n}} {\int }_{0}^{+ ∞} \{ {[ {J}_{1} ( x ) ]}^{2} erf ( x \sqrt{\frac{t {α}_{skin}}{{R}^{2}}} ) \} \frac{d x}{{x}^{2}} .$$

Referring the design with the sensor separate from the heater as design 1 and the one with sensor/heater being the same as design 2, the simplified model shows that the sensitivity of temperature change to skin water content of design 1 is about 30% smaller than that of design 2 (panel A of FIG. 11).

### Measuring Skin Water Content

In previous reports, fitting procedures based on finite element analysis (FEA) and measurements of ΔT vs. t define local, volumetric-averaged values of k for the sample under test, appropriate for homogeneous, isotropic materials (panels F-G of FIG. 2). Skin, however, is a layered structure that includes different features and heterogeneities. The outermost layer (i.e., the epidermis and its surface coating, known as the stratum corneum) contains water embedded within cells and in the tissue matrix. The dermis, the layer beneath the epidermis, contains this form of water as well as that associated with blood vessels (panel A of FIG. 3). Modeling the skin as a two-layer system enables extraction of the approximate, averaged thermal properties of the epidermis and dermis. FEA simulations relate the SHS measurements (ΔT vs. t) to the thermal properties of skin. A micromechanics model that treats the skin as a composite of dry tissue and water relates the thermal properties directly to the skin water content (φ) (panel B of FIG. 3). The FEA model assumes a thickness h for the epidermis (based on body location, Table 2), with an approximate value of about 100 μm for most locations except for acral surfaces such as the heel (h≈600 μm) and palm. At the microscale, the model for the epidermis includes a composite of dry skin (k_{dry}=0.2 W/(m-K), α_{dry}=0.15 mm²/s) and water (kw 0.6 W/(m-K), aw 0.14 mm²/s). In this way, the equivalent thermal properties of the epidermis can be related to the volumetric epidermal water content φ_{E} according to

$$\frac{{k}_{E}}{{k}_{dry}} = \frac{( p + 2 ) + 2 ( p - 1 ) {φ}_{E}}{( p + 2 ) - ( p - 1 ) {φ}_{E}} , \qquad ( 1 )$$

$$\frac{{α}_{E}}{{α}_{dry}} = \frac{{α}_{w} {k}_{E}}{( 1 - {φ}_{E} ) {α}_{w} {k}_{d r y} + {φ}_{E} {α}_{dry} {k}_{W}} , \qquad ( 2 )$$

where

$$p = \frac{{k}_{w}}{{k}_{dry}} ,$$

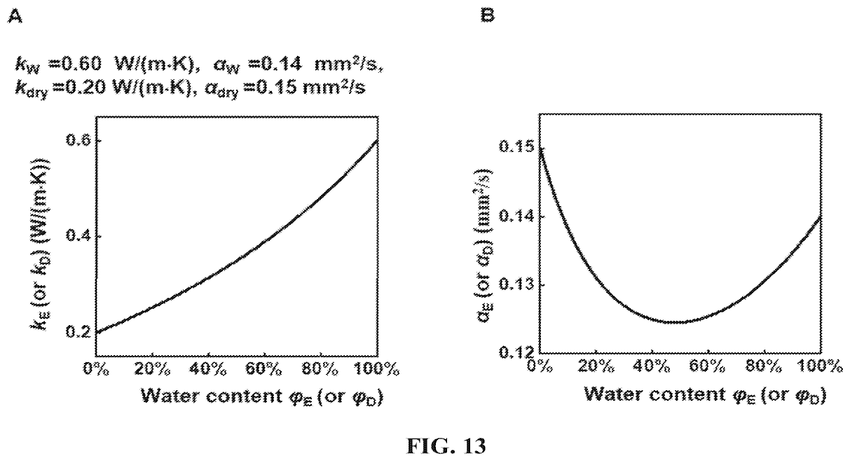

as plotted in FIG. 13, and k_{E} and α_{E} correspond to the thermal conductivity and diffusivity of the epidermis, respectively. A similar simple model applies to the dermis, such that its equivalent thermal properties are given in Eqs. (1) and (2) with k_{E}, α_{E} and φ_{E} replaced by k_{D}, α_{D} and φ_{D}, respectively.

At the macroscale, FEA establishes a relationship between ΔT (at time t=0-13 s) and the thermal properties k_{E}, α_{E}, k_{D} and α_{D}, and therefore the water content φ_{E} and φ_{D}. For the typical epidermal thickness (h=100 μm), at short times following initiation of heating (e.g., t=2 s), thermal transport occurs substantially into the epidermis but only slightly into the dermis (panel C of FIG. 3). Under these circumstances, ΔT is much more sensitive to φ_{E} than φ_{D} (panel E of FIG. 3). Conversely, at long times (e.g., t=13 s), the heat passes through the epidermis and significantly into the dermis (panel D of FIG. 3). As the dermis is much thicker than the epidermis, ΔT in this regime is much more sensitive to φ_{D} than φ_{E} (panel E of FIG. 3). This temporal separation allows separate determination of φ_{E} an D from the ΔT vs. t curves, thereby enabling real-time display of φ_{E} and φ_{D} on the phone application shortly after completing the measurement.

To determine the water content φ_{E} (epidermis) and φ_{D} (dermis) from an experimental curve of temperature change ΔT vs. time t, φ_{E} and ED are extracted through minimization of the quantity below

$$\frac{1}{{t}_{total}} {\int }_{0}^{{t}_{total}} {[ Δ T ( t ) - Δ \tilde{T} ( t ; {φ}_{E} , {φ}_{D} ) ]}^{2} dt ,$$

where tₜₒₜₐₗ is the total heating time, ΔT (t) is the temperature predicted by the FEA and micromechanics model, and ΔT(t) is the experimental measurement.

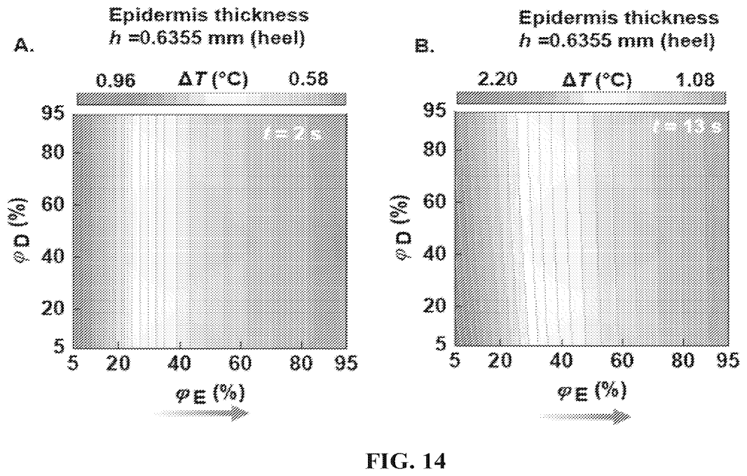

For a few cases, depending on the individual and the body location, large epidermal thicknesses (e.g., heel, h≈600 μm) limit the transport of heat to the dermis even at t=13 s. This behavior leads to a relative insensitivity of ΔT to φ_{D} (FIG. 14), such that φ_{D} is indeterminate.

Error in the determined φ_{E} and φ_{D} from error in the temperature measurement: Given a curve of ΔT vs. t, φ_{E} and φ_{D} can be determined by the fitting method shown above. Adding an error to the temperature change such that ΔT becomes ΔT(1+δ) and performing the fitting again, the differences between the fitted water contents are the errors arising from the relative error in temperature measurement δ.

Error in the determined φ_{E} and φ_{D} from error in epidermis thickness. Epidermis thickness has a small effect on the relationship of ΔT vs. φ_{E} and D. For a typical epidermis thickness h=100 μm, fitting is performed to obtain φ_{E} and φ_{D} from the experimental curve of ΔT vs. t. Changing the epidermis thickness by ±20% and performing the fitting again for the same experimental curve, the differences between the fitted water contents are the errors arising from the error in epidermis thickness. The average errors for φ_{E} and ED ranging from 5% to 95% are 2.9% for epidermis and 1.9% for dermis (for 20% error in epidermis thickness).

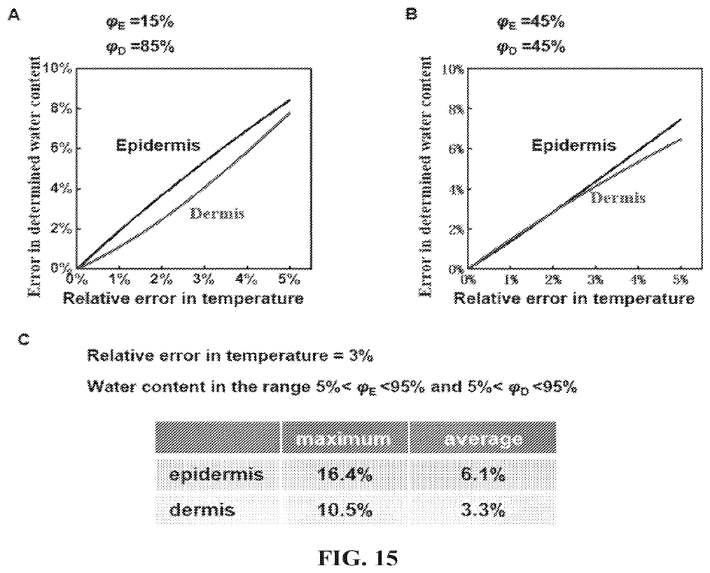

In practice, for the cases reported here, errors in the values of φ_{E} and φ_{D} determined in this manner are less than about 500 (FIG. 15). The main contributions to these errors are in the noise associated with the measurement of ΔT (less than about 3%) and in variations in h (less than about 20%, FIG. 15). Measurements on a benchtop model including mixtures of glycerin and water agree well with results from the FEA model developed here (FIG. 10).

TABLE 2
Thickness of the epidermis across various body
locations (Y. Lee and K. Hwang, Skin thickness of Korean adults.
Surgical and Radiologic Anatomy 24, 183-189, 2002). For locations
with epidermal thickness not explicitly stated in Y. Lee
and K. Hwang (wrist, leg/knee, hand/thumb, elbow,
shoulder, stomach), computations of hydration levels utilized
the epidermal thickness of an adjacent location.

| | Male (μm) | Female (μm) |
|---|---|---|
| Forearm & Wrist | 67.8 | 80.3 |
| Forehead & fossa | 95.9 | 90.4 |
| Cheek | 115.4 | 85.0 |
| Shin & Leg & Knee | 100.6 | 78.3 |
| Calf | 124.4 | 116.2 |
| Heel | 792.8 | 478.1 |
| Buttock | 147.8 | N.A. |
| Hand & Thumb | 246.8 | N.A. |
| Elbow | 112.7 | 97.1 |
| Back | 88.1 | 59.6 |
| Palm | N.A. | 647.4 |
| Shoulder | 101.2 | N.A. |
| Stomach | N.A. | 79.9 |

N.A. indicates no test performed on the gender/location.

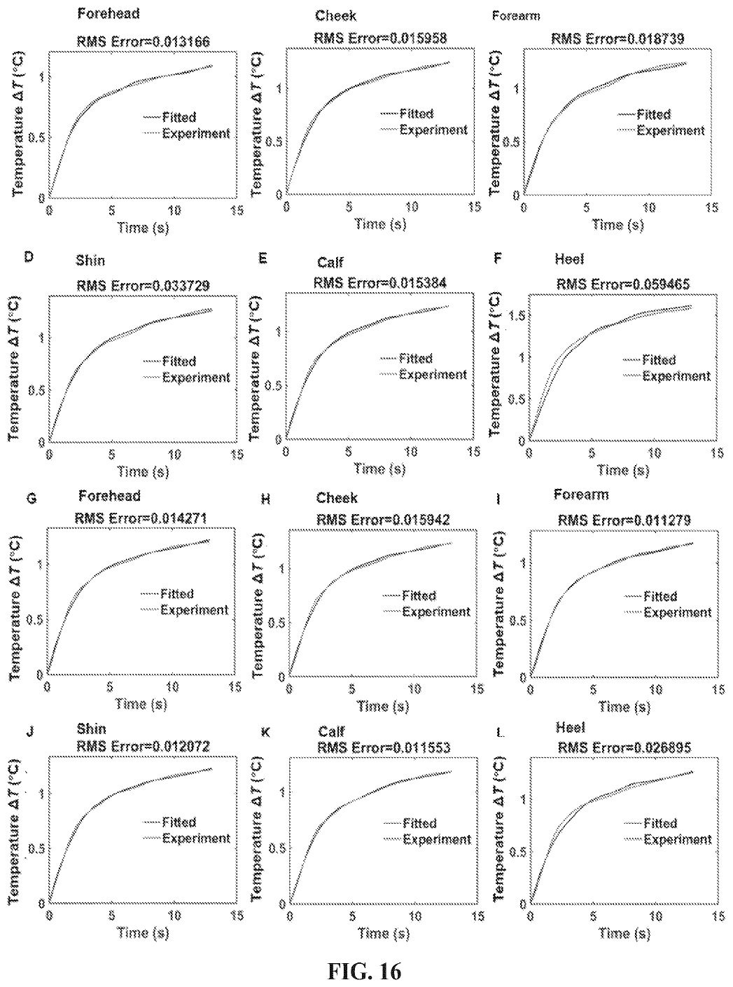

Measurements on several healthy/normal subjects (nₚₐₜᵢₑₙₜₛ=16, demographics in Table 3) on six different body locations each illustrate the accuracy of the fitting process and utility of the model (panel G of FIG. 3; FEA fits with error values in FIG. 16). φ_{E} has a larger variance than φ_{D} across the various body locations, attributed to the direct exposure of the epidermis to the environment and insulation of the dermis from environmental conditions. Furthermore, φ_{E} for the forehead and cheek show greater variations than that of other body locations likely due to the presence of sebum, an oily, waxy substance excreted by sebaceous glands (with highest density on the face and scalp) largely comprising triglycerides, fatty acids, and wax esters, all which exhibit relatively low k (0.13-0.2 W/(m-K)). Measurements across the various body locations yield a combined, average water content (including both φ_{E} and φ_{D}) for local areas (sans the heel) on healthy/normal subjects of about 63%, consistent with the literature. Typically, φ_{D}>φ_{E} (P=0.0009, n_{locations}=79), as expected due to transepidermal water loss in the epidermis and the presence of blood vessels in the dermis. This finding agrees with reports in literature, which show lower average values of water content in the epidermis (about 25% to 70%) than in the dermis (about 70%). These results validate that the model for skin hydration developed are consistent with known skin physiology.

TABLE 3
Demographics of Healthy/Normal Subjects.

| Subject | Sex | Age | Race/Ethnicity |
|---|---|---|---|
| 1 | F | 24 | Asian |
| 2 | F | 30 | White |
| 3 | F | 19 | Asian |
| 4 | M | 30 | Asian |
| 5 | F | 20 | White |
| 6 | F | 20 | Asian |
| 7 | F | 19 | White |
| 8 | M | 24 | Asian |
| 9 | F | 19 | White |
| 10 | M | 24 | Asian |
| 11 | F | 19 | Asian |
| 12 | M | 24 | Asian |
| 13 | F | 21 | White |
| 14 | M | 25 | White |
| 15 | F | 20 | White |
| 16 | M | 20 | White |
| 17 | M | 20 | Asian |

### Measurements on Atopic Dermatitis Patients

Use of the SHS system, for the first time, on patients diagnosed with various skin diseases are the key set of results which illustrate the clinical utility and the versatility of the technology. For assessments of disease lesions, φ_{E} and φ_{D}, along with skin surface temperature T₀, are important indicators of different conditions, such as edema and erythema. It is noted that calculations of φ_{E} and φ_{D} involve measurements of ΔT and are thus independent of T₀. The same types of information can also serve as quantitative metrics of the efficacy of treatment strategies, for example, moisturizers/ointments. These combined features aid in diagnosis, treatment response tracking, and potential detection of flares. The following also compares measurements of the SHS with those of a commercially available, portable corneometer (gpskin barrier pro, GPower, KR) that determines both transepidermal water loss (TEWL, a contact-less measurement using humidity and temperature sensors) and stratum corneum hydration (ScH, an electrical impedance/capacitive measurement), and shows good correlation to clinical gold-standard tewameters and corneometers. TEWL is a complementary measurement to skin permeability/hydration and assesses skin barrier function. It is noted that the corneometer, along with the aforementioned gold-standard devices, involves only superficial measurements depths, typically confined to the stratum corneum (about 15 μm). Few devices have mm-scale measurement depths but have large (1-5 cm) probe diameters which prevent small-area measurements.

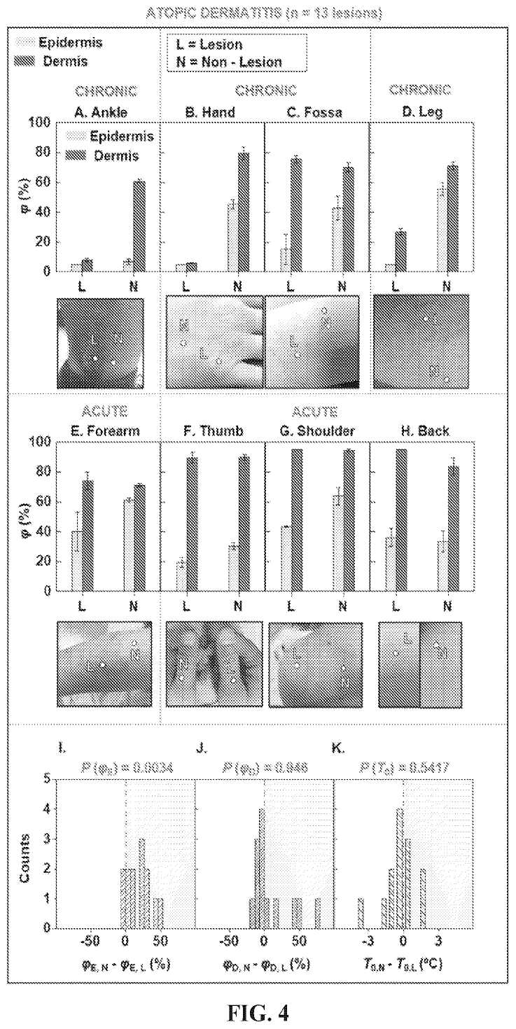

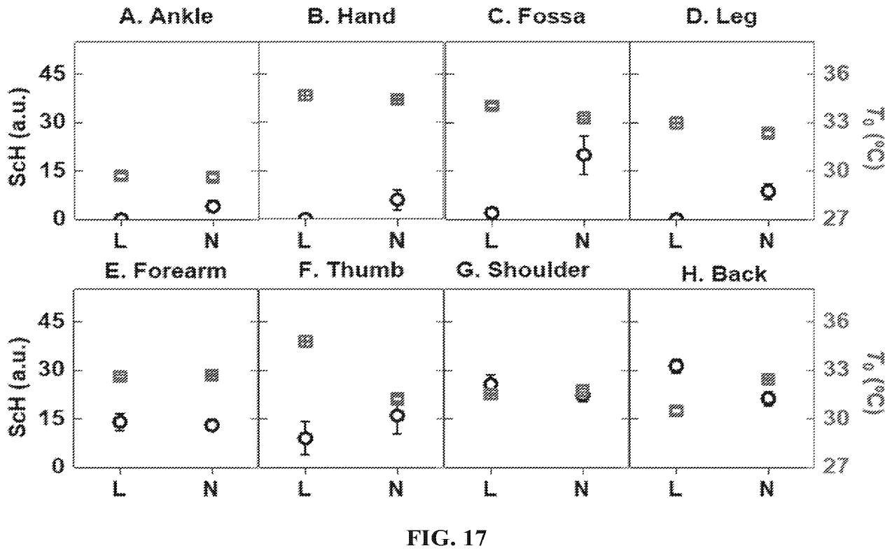

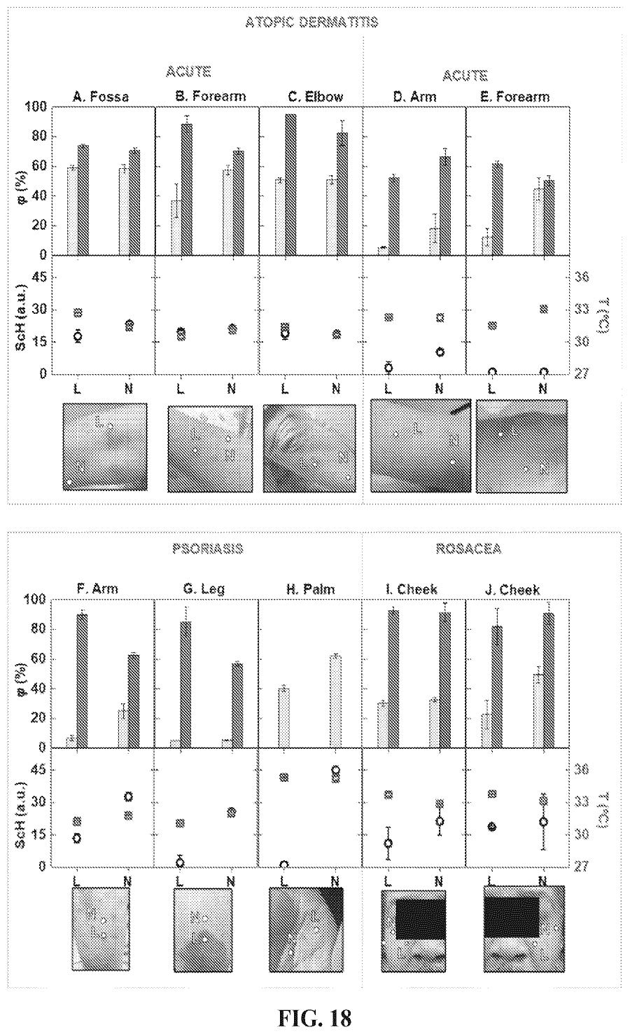

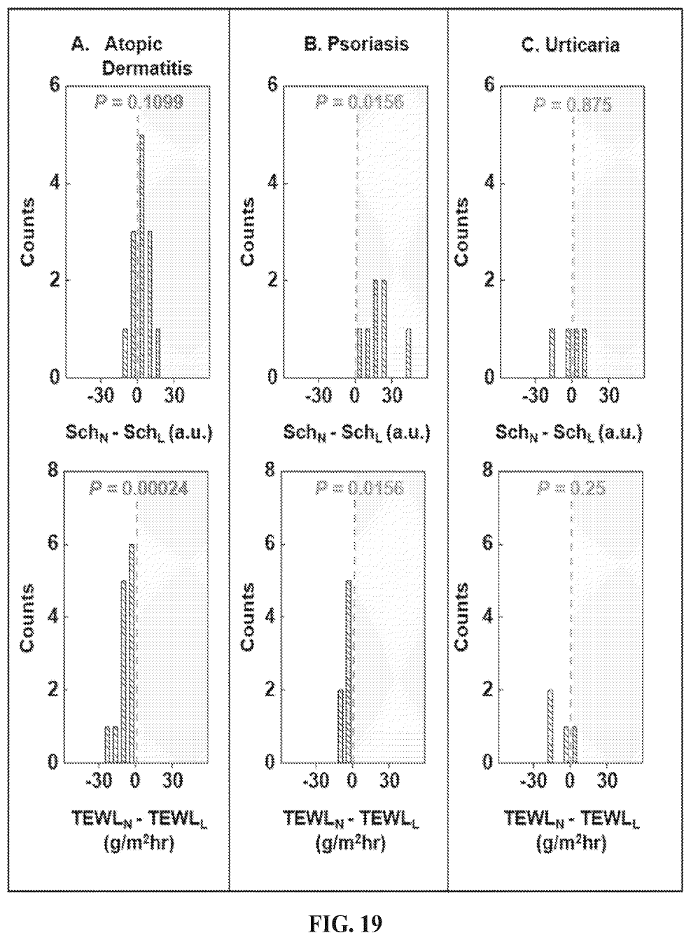

The first demonstration focuses on patients with atopic dermatitis (AD), an inflammatory disease of the skin that causes red, itchy rashes on various locations of the body. In the United States, 18 million adults suffer from AD, with worldwide prevalence rates of 1-20%. This pilot study involves nₗₑₛᵢₒₙ=13 lesions across nₚₐₜᵢₑₙₜ=7 patients clinically diagnosed with AD by a dermatologist using both the SHS and the corneometer. Patient demographics is listed in Table 4. SHS outputs include measurements of T₀, φ_{E, L(N)} and φ_{D, L(N)} on the diseased site (the lesion L), and on healthy-looking skin on a similar body location or perilesional area (non-lesional area N) as a standard of comparison. T₀ and corneometer data for lesions in FIG. 4 not displayed in FIG. 4 for brevity are in FIG. 17. Results for other lesions are in FIG. 18. Three patients with chronic AD (panels A-D of FIG. 4) display low values of φ_{E,L} (less than about 20%) compared to the non-lesional sites, consistent with the clinically dry appearances of the lesions. For the patients in panels A-B of FIG. 4, the φ_{D,L} is also much lower at the site of the lesion than φ_{D,N}. Instances where φ_{D,L}φ_{D,N} suggest that layers of skin below 100 μm are also drier than normal skin. A possible explanation is that the epidermis is thicker than the assumed value of 100 μm, attributed to hyperkeratosis. Panels E-H of FIG. 4 display images of acute AD lesions and their corresponding φ_{E} and φ_{D}. φ_{E,L} at the lesion is on average about 20% lower than φ_{E,N} (P=0.0034). φ_{D} and T₀ do not display significant differences between the lesional and non-lesional sites (panels I-K of FIG. 4). These results are consistent with the understanding that AD is an epidermal disease. In contrast, data obtained with the corneometer present no clear demarcation between diseased and non-lesional locations for ScH (fP=0.1099) but measurements of TEWL (P=0.00024) (FIG. 19) exhibit indications of the disease.

TABLE 4
Demographics of Diseased Patients.

| | Panel(s)/ | | | | |
|---|---|---|---|---|---|
| Subject | FIG(S). | Diagnosis | Sex | Age | Race/Ethnicity |
| 18 | 4A | Atopic Dermatitis | F | 55 | Black/African |
| | | | | | American |
| 19 | 4B, 4C | Atopic Dermatitis | M | 20 | White/Asian |
| 20 | 4D | Atopic Dermatitis | M | 19 | Asian |
| 21 | 4E | Atopic Dermatitis | F | 54 | Asian |
| 22 | 4F-4H | Atopic Dermatitis | M | 52 | Asian |
| 23 | 5A, 18F, 18G | Psoriasis | F | 52 | White |
| 24 | 5B, 5C | Psoriasis | M | 49 | White |
| 25 | 5D, 18H | Psoriasis | F | 77 | American Indian |
| 26 | 5H | Urticaria | F | 48 | Black/African |
| | | | | | American |
| 27 | 5I-5K | Urticaria | F | 37 | Asian |
| 28 | 6A-6K, 18A- | Atopic Dermatitis | F | 64 | White |
| | 18C | Xerosis Cutis | | | |
| 29 | 18D, 18E | Atopic Dermatitis | M | 48 | White |
| 30 | 18I, 18J | Rosacea | M | 45 | Black/African |
| | | | | | American |

Measurements on Patients with Psoriasis and Urticaria

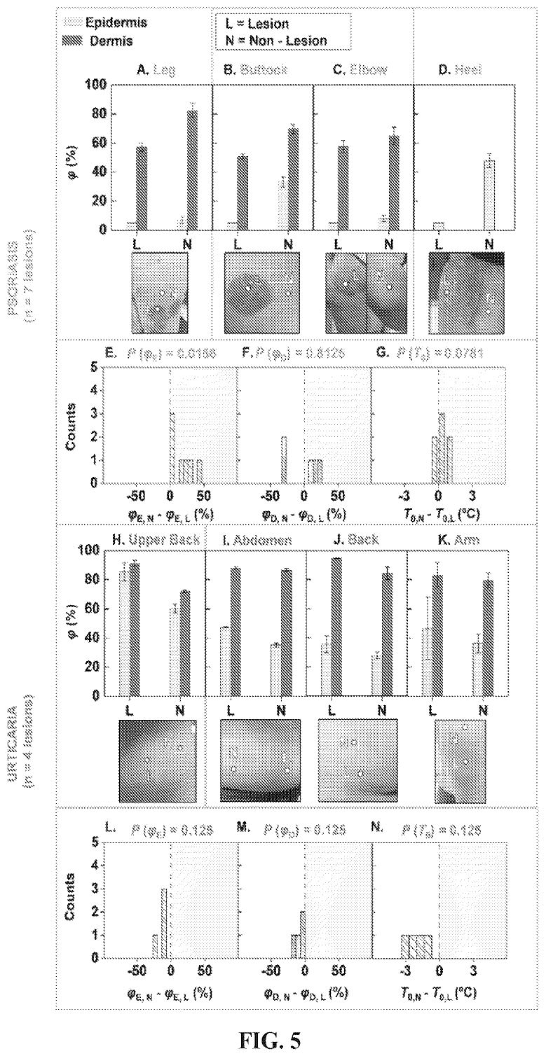

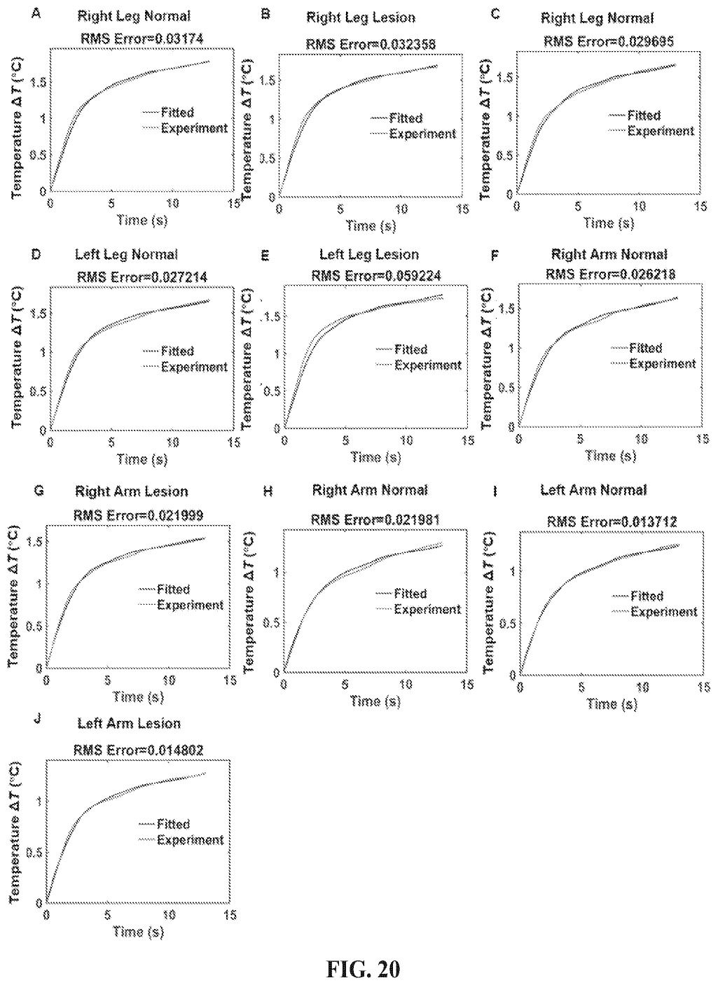

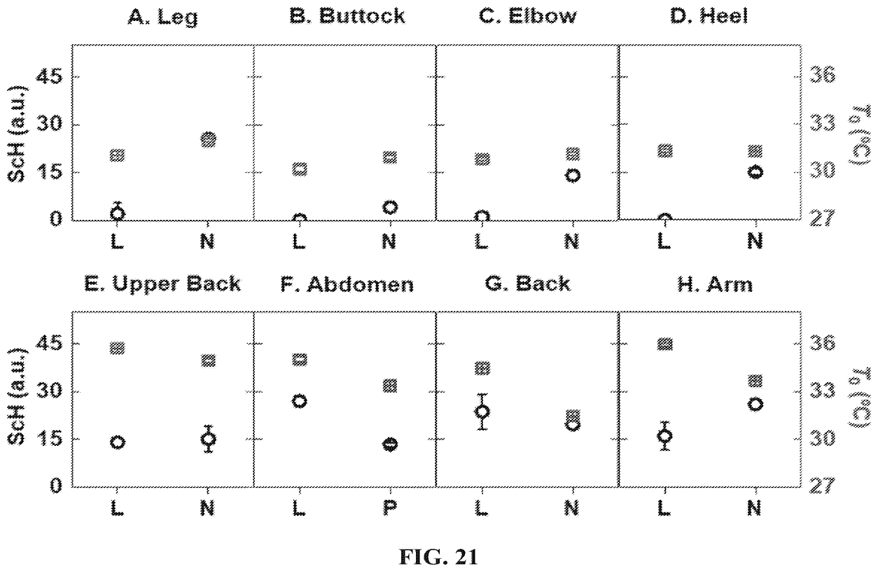

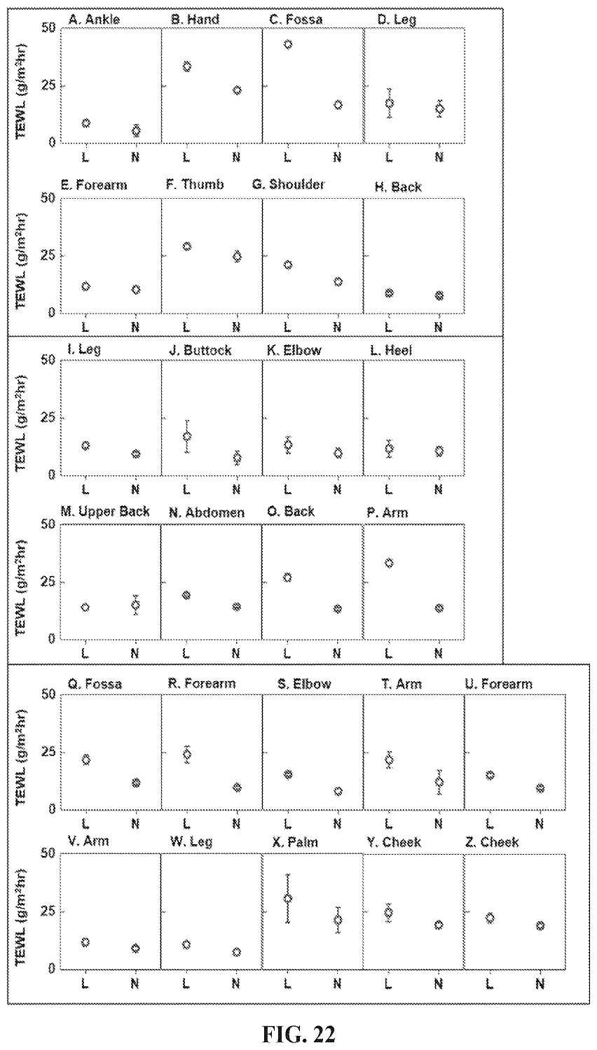

Additional clinical studies use the SHS to measure φ_{E}, φ_{D}, and T₀ (see FEA curve fits in FIG. 20, and FIG. 21 for T₀ and corneometer data) for nₗₑₛᵢₒₙ=7 lesions across nₚₐₜᵢₑₙₜ=3 patients with psoriasis a skin disease characterized by a thickened epidermis and sanguineous, scaly, plaques affecting more than 8 million people in the U.S. For the psoriasis lesions presented in panels A-D of FIG. 5, both φ_{E,L} and φ_{E,N} exhibit low values. As with certain patients with AD, φ_{D,L}<φ_{D,N}, again suggest a thickening of the epidermis, which in the corresponding images appears as white, flaking skin. In other cases (FIG. 19), φ_{D,L} is much larger than φ_{D,N}. Here, the lesions have a deep red color without the presence of peeling skin, suggesting a younger lesion with a thinner epidermis than the lesions shown in panels A-D of FIG. 5. The color suggests elevated blood flow in the dermis. Hence, for older lesions, the SHS response is likely insensitive to this flow, whereas the sensitivity should be comparatively higher for younger lesions with relatively thinner epidermis. The value of φ_{D}, therefore, may lend insight into age of psoriasis lesions, but this possibility merits further investigation on a larger number of patients. Of additional note are two unique cases of acral psoriasis (panel D of FIG. 5 and FIG. 18), where φ_{E,L}<φ_{E,N}, consistent with the flaky dehydrated appearance of the lesion compared to the perilesional location. The SHS can differentiate between psoriasis lesions and non-lesional areas with φ_{E} (P=0.0156). By contrast, differences in φ_{D} (P=0.8125) and T₀ (P=0.0781) are insignificant, confirming that psoriasis is indeed an epidermal disease (panels E-G of FIG. 5). Measurements with the corneometer also show some differences between the lesion and non-lesional area (P=0.0156 for both ScH and TEWL) (FIGS. 19 and 22).

The SHS can also provide insights into dermal diseases owing to its capability to measure properties deep into the skin, through measurements performed on patients with urticaria. This condition occurs in the epidermis and the upper portions of the dermis, where localized vasodilation occurs, and biofluid exudes into the surrounding tissue. Measurements on nₚₐₜᵢₑₙₜ=2 patients for a total of nₗₑₛᵢₒₙ=4 urticaria lesions indicate enhanced water content in the lesion than the non-lesional area for both the epidermis and dermis (panels H-M of FIG. 5). The values of T₀ for all four lesions, furthermore, are consistently larger than those of the corresponding normal location, suggesting an expected enhancement microvascular blood flow in the dermis of the lesions (panel N of FIG. 5). Similar trends in T₀ also appear in lesions associated with a patient with a different dermal disease, rosacea (FIG. 18). Due to the relatively small sample size, P is insignificant. The corneometer exhibits no trend due to its shallow measurement depth (FIG. 19). This collection of studies highlights the utility of SHS in assessing hydration across a broad range of skin diseases, with insights that extend beyond the superficial layers of skin compared to traditional skin impedance/TEWL-based devices. These findings correlate well with expected histopathology findings of both psoriasis and urticaria. The results demonstrate that the SHS can detect parameters that may serve as disease signatures—characteristic, disease-specific, quantitative trends in φ_{E}, φ_{D}, and/or T₀—for accurate, quantitative monitoring and detection of diverse dermatological conditions.

Topical Treatment on a Patient with Xerosis Cutis and Atopic Dermatitis

Diagnosing a disease state represents the first step in patient care. The next involves delivering treatment, monitoring the effects, modulating the treatment protocol as necessary, and potentially predicting for flares based on quantitative, individualized measurements on specific lesion sites. The results in the following demonstrate this approach in the context of a topical cream (CeraVe Moisturizing Cream, DFB Technology, Ltd., USA) applied to a patient diagnosed with both xerosis cutis (XC) and AD.

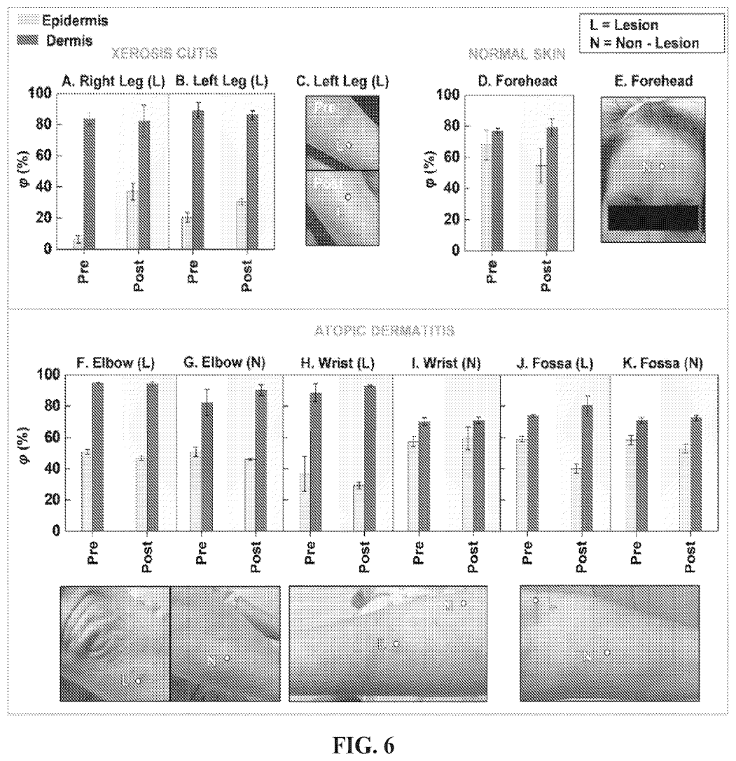

Measurements with the SHS indicate that for both XC lesions on the anterior leg, application of moisturizer for 30 minutes improves φ_{E} from its low initial value (+10 to 30%) but does not affect φ_{D}, as expected because moisturizers only improve superficial skin hydration (panels A-B of FIG. 6). Photographs of the left leg of the subject pre- and post-moisturizer visibly illustrate the improvement in hydration of the epidermis (panel C of FIG. 6). Measurements of φ on the normal skin of the forehead before and after application of the moisturizer show negligible differences, as the skin of the forehead has high baseline hydration levels and a visually hydrated appearance (panels D-E of FIG. 6).

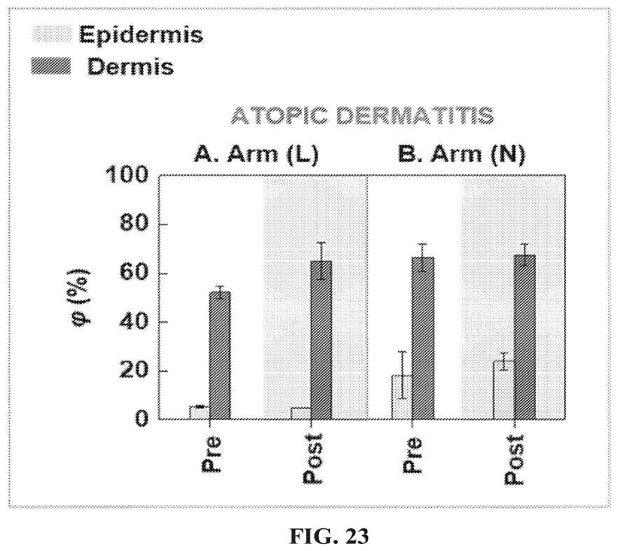

The acute AD lesions on the elbow, wrist, and antecubital fossa exhibit no increase in φ_{E,L} with moisturizer and negligible change in φ_{D,L} (panels F-K of FIG. 6). Results from a second patient treated with the same moisturizer on two AD lesions also indicate no significant changes in φ_{E,L} or φ_{E,N} (FIG. 23). Due to the weeping nature of these acute AD lesions, and because φ_{E} or φ_{D} indicate negligible differences between the lesion and non-lesional areas, this topical cream may not prove effective in improving the disease symptoms. Even for the same patient, moisturizer is effective only for certain diseases and locations. An important feature of the SHS measurement is that it probes deeply into the skin and can thus assess φ of the entire epidermis. In contrast, the corneometer, as with other skin-impedance/capacitance-based devices, has a shallow measurement depth, where measurements can be significantly influenced by the water content associated with residual moisturizer on the skin, not the skin itself. The SHS thus demonstrates potential in not only assisting in the diagnosis of skin disease, but also in determining the effectiveness of topical and systemic treatments.

### DISCUSSION

Thin, soft, wireless skin hydration sensors disclosed in this invention exhibit high repeatability in measurements, robust operation in various conditions, and, through careful systems engineering, overcome the severe shortcomings of poor repeatability in previous work. The robust design has enabled the first true application of well-known thermal physics and the TPS technique in actual clinical/non-clinical settings under varied conditions for several patients over multiple body locations. The feature of wireless data transmission and use with a wide range of mobile devices facilitates use in assisting patients with quantitative tracking of their skin health and sharing these data with their physicians. The designs specifically align with commercial manufacturing capacity that supports the consumer electronics industry, thus allowing for low-cost production and broad distribution to both clinicians and patients. This work thus has the potential to create opportunities for measurements not formerly achievable on patients in clinical environments and in the home.

Measurement areas have millimeter scale dimensions, allowing detection and mapping of small lesions on the skin, even on challenging and/or sensitive regions of the body. The measurement depths can reach about 1 mm, and the temporal information allows discrimination between surface and deep skin properties from a single set of time series data. The contact to the skin occurs naturally or can be paired with external medical-grade adhesives, without need for control over application pressure. These three features are absent from conventional corneometers. Furthermore, the device offers superior measurement capabilities, with average repeatability error of about 5% for φ_{E} and about 4% for φ_{D}. As comparison, the average error for TEWL and ScH from the commercial corneometer are about 14.4% and about 30.1%, respectively.

A limitation of the measurement is that the thickness of the epidermis is typically not known, and it cannot be easily inferred directly from the thermal data. For healthy/normal subjects, the literature values represent fair assumptions, as small variations in thickness do not alter fitting results significantly. On the other hand, for diseased locations, the thickness can change dramatically. While in these cases the top about 100 μm may not necessarily represent the ‘epidermis,’ the hydration values of the top 100 μm of skin and remaining about 900 μm below are accurate and serve the need for skin diagnostics. Paired with visual inspection, and as seen in measurements on patients with visible skin thickening, the device may yield signatures for lesions with different skin thickness. Determining signatures for each disease and sub-type warrants, however, careful investigation on many patients. Procedures include characterizing the difference between lesions and non-lesional areas, acute and chronic conditions, different subject populations, and response to treatments. The scope of the SHS platform can also be extended to monitor water content of internal organs for various diseases where traditional monitoring techniques (blood tests, MRI, etc.) fail to offer continuous assessment of organ health.

Ultimately, the technology presented here allows monitoring of skin health anywhere, anytime, and by anyone. The SHS has the potential for use in both clinical and at-home settings, from its ability to discern atopic dermatitis lesions (P=0.0034) and psoriasis lesions (P=0.0156) with high significance, detect trends for dermal diseases like urticaria and rosacea, and assess treatment efficacy. Capabilities for determining changes in skin water content presents an important benefit across the entire continuum of care for inflammatory skin diseases. This soft, conformal, battery-free system device may provide significant perspectives on skin diseases and continuous monitoring of skin health.

### Example 2

### Novel Wearable Sensor for the Objective Determination of Skin Barrier Function: Clinical Validation in a Single Arm, Open-Label, Cohort Study of ΔTopic Dermatitis, Xerosis Cutis, and Healthy Normal Subjects

The skin serves a variety of roles as an immune, sensory, and thermoregulatory organ, but one could argue its function as a protective barrier is the most fundamental. Atopic dermatitis (AD) and xerosis cutis (XC) are two common skin conditions that demonstrate skin barrier dysfunction through itch and dry skin. While both AD and XC are characterized by impaired skin barrier function, the pathogenesis is distinct.

Atopic dermatitis is recognized as a chronic inflammatory disease of the skin. Affecting up to 20% of children and 3% of adults, the most common and significant symptoms are dry skin and itch, but lesions, severity, and course are all highly variable among patients with management aimed at symptomatic relief and prevention of flares. Although it has been known that damage to the epidermis was a component of AD, recognition that abnormalities of the skin barrier as contributory in pathogenesis is more recent. Intrinsic barrier factors that contribute to dysfunction in AD are believed to be diverse, including inherited filaggrin abnormalities, decreased ceramides, and disordered tight junctions; the effect of these factors is compounded by autoimmune pathways, including action of T-helper type 2 cells and their related cytokines, as well as external factors such as exposure to environments with low humidity. Ultimately, patients with AD have decreased stratum corneum hydration, increased trans-epidermal water loss (TEWL), and higher pH when compared to non-atopic individuals.

While the epidemiology of xerosis cutis is difficult to assess due to its broad definition and wide range of severity, it is recognized as particularly common in older patients with some estimates up to 86% of patients over the age of 65. Given the high occurrence of itch in patients with xerosis cutis, there is also a higher risk of complications resulting from scratching, including pain, infection, and overall diminished quality of life due to impaired ability to participate in daily activities. Regarding the pathophysiology of XC, it is most commonly the result of age-related changes in the physiology of the skin barrier, namely the ability of the skin to generate and retain natural moisture, proteins, and lipids. Furthermore, UV radiation leading to natural breakdown of filaggrin and reduced levels of natural moisturizing factor is also contributory with cumulative effect over multiple exposures.

While there are existing physician-grading scales, there are currently no widely used objective digital biomarkers that accurately reflect the severity of the disease or the clinical manifestations of AD and XC. Thus, there is need for objective and repeatable methods to assess skin barrier function in normal and affected skins. Subjective grading and patient reported outcomes are indirect methods of assessing the health of the skin barrier that are commonly used to guide management. Examples of scoring systems include the overall dry skin (ODS) score, six area six sign (SASSAD) score, and the AD area severity index (ADASI). However, these grading systems are imprecise due to user variability and can be unreliable for assessing small but clinically meaningful changes over time. These measures also become difficult to apply in populations such as pediatrics where patients may be unable to adequately report the severity of their symptoms. These limitations were the reason for developing objective measurements such as skin hydration and TEWL to quantify the health of the skin barrier.

Traditionally, corneometry has been used as an objective measure, relating skin impedance to the water content of the stratum corneum. There has also been work demonstrating the use of spectroscopy as a means of evaluating skin hydration. TEWL, or the measure of water passively evaporating through the skin to the external environment, is also generally considered to be an indicator of skin barrier health, but the accuracy is often influenced by environmental factors like humidity and temperature. However, well-researched tools that are currently available for measuring skin hydration and TEWL are generally bulky, expensive, and have limitations in terms of repeatability and generalizability.

In this exemplary study, we present the findings from a clinical validation study of a novel skin hydration sensor (SHS) to address existing technology limitations, and demonstrate the ability for the SHS to capture measurements of skin barrier function in a diverse population including healthy normal subjects, and patients with atopic dermatitis and xerosis cutis in comparison to a clinician-derived ODS score. The SHS is a wireless, flexible sensor that evaluates the thermal conductivity of the skin to determine water content. The use of thermal conductivity is a novel approach to evaluating skin hydration that has been established as a direct means of measuring skin hydration as a function of skin barrier health. This modality also offers the advantage of overcoming depth limitations seen with impedance-based devices. Prior validation studies have also established that measurements with the SHS device are more repeatable and less susceptible to ambient changes in temperature than earlier attempts to leverage thermal conductivity in evaluating hydration. In addition, the form factor of the devices allows for flexible conformation to the skin that improves skin contact, patient comfort, and even continuous wear. The device is also supported by wireless communication capabilities with a wide range of mobile devices allowing for assessments in a patient's natural home environment.

Overall, the SHS device provides objective measurements of skin hydration barrier function with clear discrimination between different ODS scores across three body locations and the application of a moisturizer. Furthermore, the SHS device provides the ability to distinguish skin barrier function across healthy volunteers, XD, and AD patients. This device may be useful as an endpoint for clinical trials for experimental therapeutics, assess treatment response in clinical practice, and warn of impending deterioration of skin barrier function prior to worsening clinical symptoms.

### Devices

The SHS device developed at Northwestern University is described above. The device in one embodiment includes a 1 by 1.5 cm flexible sensor coated in silicone. The device applies a small amount of heat to the skin and uses temperature sensors to calculate the thermal conductivity of the skin, which corresponds with water content. Measurements are displayed through an app connected via Bluetooth®.

### Participants

This study was approved by the institutional review boards of Ann and Robert H. Lurie Children's Hospital of Chicago and Northwestern University.

Participants were recruited in the outpatient dermatology clinic of Northwestern Medicine. Written, informed consent was obtained prior to study participation. Exclusion criteria were: <18 years of age, pregnancy, and active, open lesions or other dermatologic history that precluded sensor placement. Dermatologic history was obtained via electronic chart review. A total of 136 subjects (86 women and 50 men) with a mean age of 44 years (range of 19-95 years, standard deviation of 19 years) were enrolled in this study including 18 patients with AD. 40% of subjects self-identified as White/Caucasian, 31% of subjects self-identified as Asian, 14% of subjects self-identified as Black/African American, and 8% of subjects self-identified as Hispanic/Latino. A summary of the demographics in this study is included in Table 5. 139 measurement sites with XC, and 215 healthy normal measurement sites. We correlate the objective measurements captured by the SHS sensor with a dermoscopic and clinical image derived ODS scores between two graders across all subjects in three body locations (forearm, lower leg, and forehead). A subgroup of participants were assessed with the SHS sensor before and after moisturizer application (n=49 subjects). A Kruskal-Wallis test with multi-comparison correction was conducted in a post-hoc analysis comparing the SHS measurements organized by clinician-derived ODS scores.

TABLE 5
Overall demographics of participants in the study.

| | | Age |
|---|---|---|
| | | (mean age ± standard |
| | n (%) | deviation in years) |
| Overall | 136 (100) | 44 ± 19 |
| Male | 50 (37) | 48 ± 19 |
| Female | 86 (63) | 42 ± 18 |
| Asian | 42 (31) | 33 ± 12 |
| Black/African American | 19 (14) | 54 ± 17 |
| Hispanic/Latino | 11 (8) | 37 ± 13 |
| White/Caucasian | 55 (40) | 52 ± 19 |
| Unknown/Not Reported | 9 (7) | 43 ± 19 |

### Study Procedures

Anatomic sites selected for this study included the flexor surface of the forearm, the flexor surface of the lower leg, and the midline of the forehead. These areas were chosen primarily for accessibility. The forehead was selected as an internal control known to have consistently higher hydration compared to other sites. Specific placement of the devices was chosen to avoid subject-specific factors (e.g., body hair, cuts, tattoos). A 5×5 cm area at each anatomic site was identified, sanitized with a single-use alcohol wipe, and marked with a surgical marker to ensure that the same location was used for all measurements.

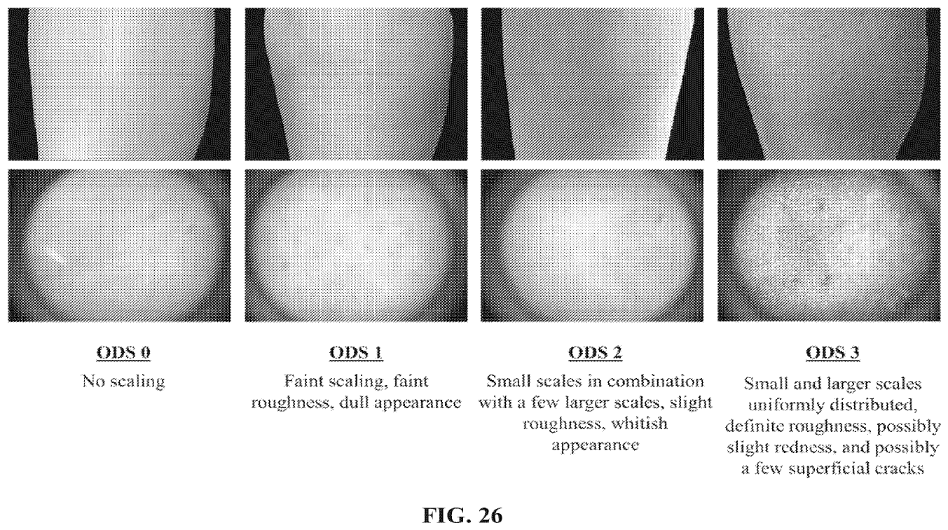

Study visits began with a set of photographs of the measurement sites. One set of photographs was taken with an iPhone X camera, and one set was taken with an iPhone X camera and a Dermlite dermatoscope attachment. These photographs were used to issue an overall dry skin score to the site. Images were scored by two reviewers with an aim of 90% concordance. A third reviewer was brought in to decide between differing scores. Of note, ODS 4 was not used in this study because the definition for this score is “dominated by large scales, advanced roughness, redness present, eczematous changes and cracks” and active, open lesions or cracking of the skin was an exclusion criteria. FIG. 26 shows ODS scoring criteria with examples.

Triplicate measurements were recorded with the SHS at each measurement site. Single-use alcohol wipes were used to sterilize the devices.

### Optional Moisturizer Extension

An optional extension of the study was also offered to participants. In this extension, 50 mg of moisturizer was gently applied to the sites of interest and rubbed into the skin for 15 seconds. These application parameters were selected for consistency with existing sunscreen studies as well as the FDA recommendation for sunscreen application (2 mg/cm²). Another set of measurements (triplicate at each location with each device) were then taken 15 minutes after application of the moisturizer. The standard commercial moisturizer used was an aqueous cream manufactured by CeraVe.

### Statistical Analysis

Results were analyzed and visualized in MATLAB®. The differences in measurements across ODS scores, anatomic locations, and skin condition (i.e. healthy control, XC, AD) were analyzed using a nonparametric Kruskal-Wallis with Tukey post-hoc analysis. A Wilcoxon rank sum test was used to compare the measurements taken before and after the application of moisturizer. While not a primary goal of the program, concurrent corneometry measurements (Delfin Moisture Meter) were also obtained. Prior work has demonstrated greater repeatability of the SHS sensor versus corneometry.

### Results

Skin hydration readings (a scale of 0 to 1) decreased with increasing ODS at each site (p<0.001, p=0.003, and p<0.001 for the arm, leg, and forehead, respectively). Median measurement at ODS 0 vs. ODS 3 at the arm were 0.614 (CI: 0.601-0.627) vs. 0.3858 (CI: 0.356-0.415) and 0.6525 (CI: 0.631-0.675) vs. 0.3642 (CI: 0.341-0.388) for ODS 0 vs. ODS 3 at the leg. In the moisturizer subgroup, a baseline ODS score of 3 yielded a greater increase (66% and 41% at the arm and leg, respectively) in skin hydration after moisturizer application compared to baseline ODS scores of 2 (33% and 22% at the arm and leg, respectively), 1 (18% and 13% at the arm and leg, respectively), or 0 (5% and 2% at the arm and leg, respectively). Median measurement at the arm and leg were higher for HNL measurement sites 0.6070 and 0.6501 for the arm and leg, respectively) vs. sites designated XC (0.5238 and 0.4871 at the arm and leg, p<0.001 at both the arm and leg when compared to HNL) or AD (0.6781 and 0.7224 at the arm and leg, p=0.09 at the arm and p=0.48 at the leg).

Comparison of SHS Measurements with ODS Scores

SHS measurements range from 0 to 1, calibrated with standardized silicone models with known hydration levels. ODS scores ranged from 0-3 for the arm and leg. No subjects received an ODS score of 2 or higher for the forehead. Median SHS measurements at the arm were 0.6140 (95% CI: 0.6006-0.6274, n=66), 0.5540 (CI: 0.5340-0.5741, n=43), 0.4832 (CI: 0.4246-0.4923, n=14) and 0.3858 (CI: 0.3563-0.4154, n=3) for ODS 0, 1, 2, and 3, respectively. Comparison by Kruskal Wallis yielded p<0.001 for all comparisons, with the exception of between 2 and 3 (p=0.04). Median SHS measurements at the leg were 0.6525 (CI: 0.6305-0.6745, n=37), 0.5955 (CI: 0.5631-0.6278, n=23), 0.5331 (CI: 0.5103-0.5559, n=44) and 0.3642 (CI: 0.3406-0.3878, n=32) for ODS 0, 1, 2, and 3, respectively. Comparison by Kruskal Wallis yielded p<0.001 for all comparisons, with the exception of between 0 and 1 (p=0.03). Median SHS measurements at the forehead were 0.6898 (CI: 0.6811-0.6985, n=133) and 0.4635 (CI: 0.3242-0.6029, n=3) for ODS 0 and 1, respectively with p=0.003 by Kruskal Wallis comparison.

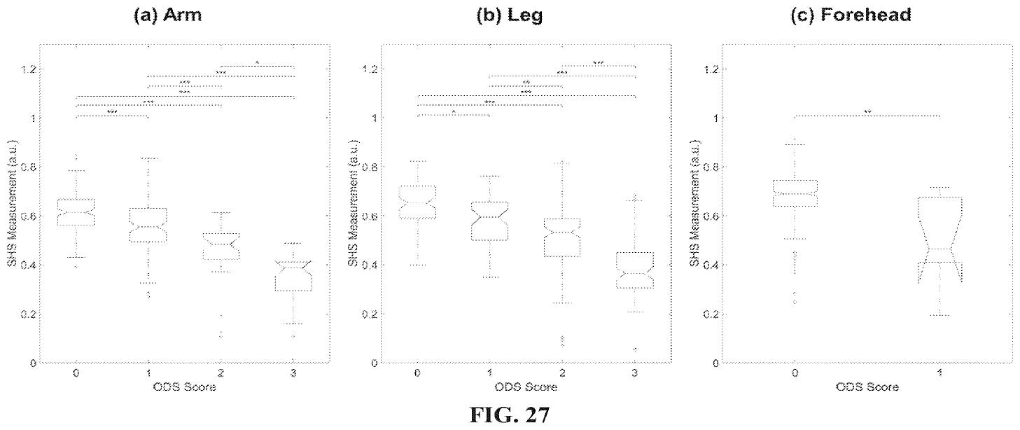

At all sites, a linear relationship can be observed, with a 37.2% and 44.2% relative decrease in median measurement when comparing ODS 0 to ODS 3 at the arm and leg, respectively. A 32.8% relative decrease in median measurement was seen at the forehead from ODS 0 to ODS 1. Of note, 133 of 136 forehead images in this study were consistent with an ODS score of 0. The forehead was found to have higher hydration than arm (p<0.001) or leg (p=0.001) at ODS 0 by Kruskal Wallis. Mean hydration readings for ODS 0 were 0.6175, 0.6476, and 0.6842 at the arm, leg, and forehead, respectively. FIG. 27 displays the comparison of SHS measurements and ODS scores, separated by location. Table 6 in supplementary materials shows the number of subjects with each ODS score at each location.

TABLE 6
Mean SHS measurements organized by ODS score and anatomic
location in the overall study. Measurements taken after moisturizer
application in the extension of the study are not included

| ODS | Arm | Leg | Forehead |
|---|---|---|---|
| 0 | 0.6175 (n = 66) | 0.6476 (n = 37) | 0.6842 (n = 133) |
| 1 | 0.5552 (n = 43) | 0.5812 (n = 23) | 0.5115 (n = 3) |
| 2 | 0.4599 (n = 14) | 0.5057 (n = 44) | — |
| 3 | 0.3390 (n = 13) | 0.3811 (n = 32) | — |

### Comparison of SHS Measurements Taken Before and After Moisturizer Application

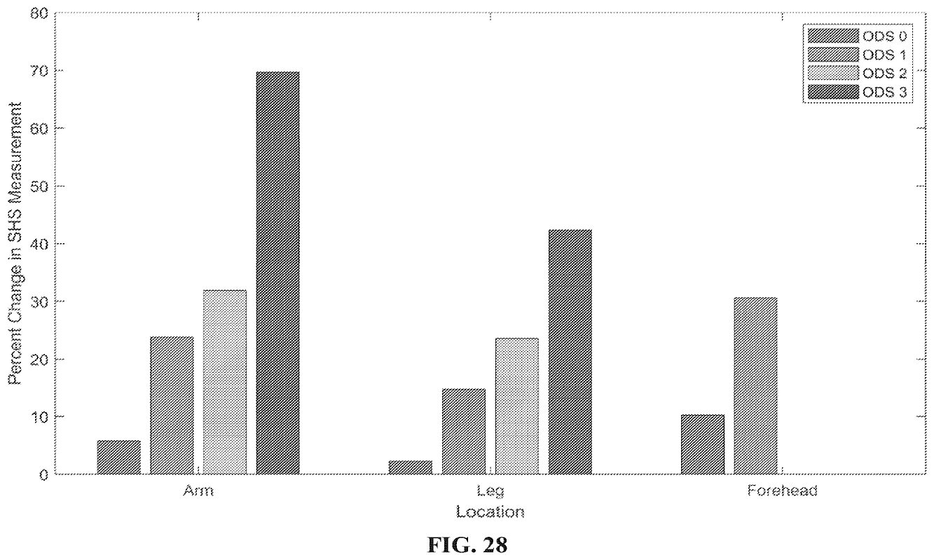

49 subjects participated in the moisturizer extension of the study. One subject was excluded from the forehead analysis due to technical failure while collecting post-moisturizer measurements. FIG. 28 displays the mean change in SHS measurement by ODS score and location after moisturizer application. In the moisturizer subgroup, a baseline ODS score of 3 yielded a greater increase in skin hydration after moisturizer application compared to baseline ODS scores of 0, 1, or 2. The mean change in SHS measurement after moisturizer application was highest at ODS 3 (0.2246 and 0.1565 at the arm and leg, respectively), compared to ODS 2 (arm: 0.1518, leg: 0.1124) ODS 1 (arm: 0.1011, leg: 0.0747), or ODS 0 (arm: 0.0297, leg: 0.0129). The mean percent increase in SHS measurement was statistically significant for ODS 1 (18%, p<0.001), ODS 2 (33%, p=0.004) and ODS 3 (66%, p=0.004) at the arm and for ODS 2 (22%, p=0.006) and ODS 3 (41%, p<0.001) at the leg by Wilcoxon rank sum test, details in Table 7 in supplementary materials. Application of moisturizer did not result in statistically significant change in forehead measurement (6% with p=0.06 for ODS 0 and 24% with p=0.35 for ODS 1).

TABLE 7
Mean SHS measurements obtained in the moisturizer extension
of the study. P values compare measurements taken before vs. after
moisturizer application. Total number of
participants in the moisturizer extension of the study
was 49, one subject was excluded from forehead analysis due to
technical failure in obtaining post-moisturizer measurements.

| ODS | | 0 | 1 | 2 | 3 |
|---|---|---|---|---|---|
| Arm | Baseline | 0.6237 | 0.5430 | 0.4922 | 0.3298 |
| | After | 0.6534 | 0.6441 | 0.6440 | 0.5544 |
| | Moisturizer | | | | |
| | p value (n) | 0.10 (17) | <0.001 (22) | 0.004 (5) | 0.004 (5) |
| Leg | Baseline | 0.6822 | 0.5359 | 0.5071 | 0.4336 |
| | After | 0.6951 | 0.6106 | 0.6195 | 0.5901 |
| | Moisturizer | | | | |
| | p value (n) | 0.36 (13) | 0.10 (8) | 0.006 (11) | 0.001 (17) |
| Fore- | Baseline | 0.6539 | 0.5115 | | |
| head | After | 0.7035 | 0.6337 | | |
| | Moisturizer | | | | |
| | p value (n) | 0.06 (45) | 0.35 (3) | | |

### Comparison of SHS Measurements by Condition

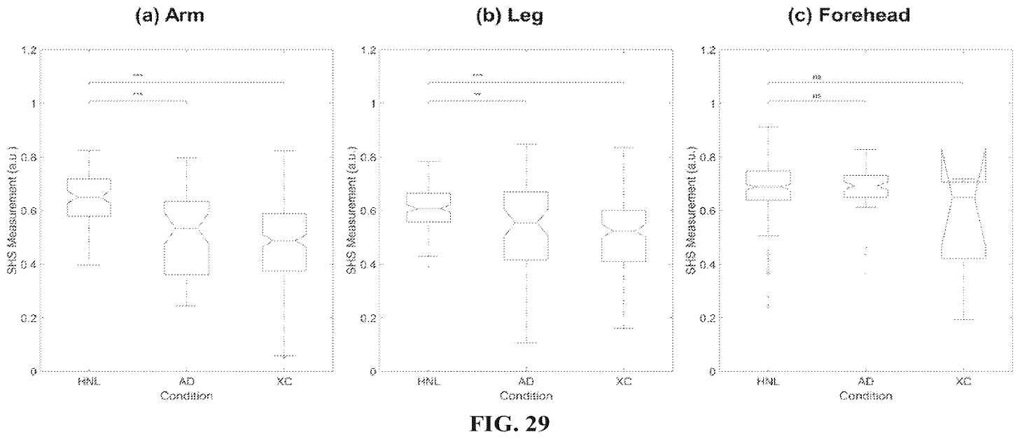

A comparison of SHS measurements organized by skin conditions is shown in FIG. 29. The HNL and XC groups were distinguished by ODS scores. Measurement sites with an ODS of 0 were placed in the HNL group for analysis while ODS values of 1 or greater were placed in the XC group. Measurements from subjects with known diagnosis of AD were placed in the AD group regardless of ODS scores.

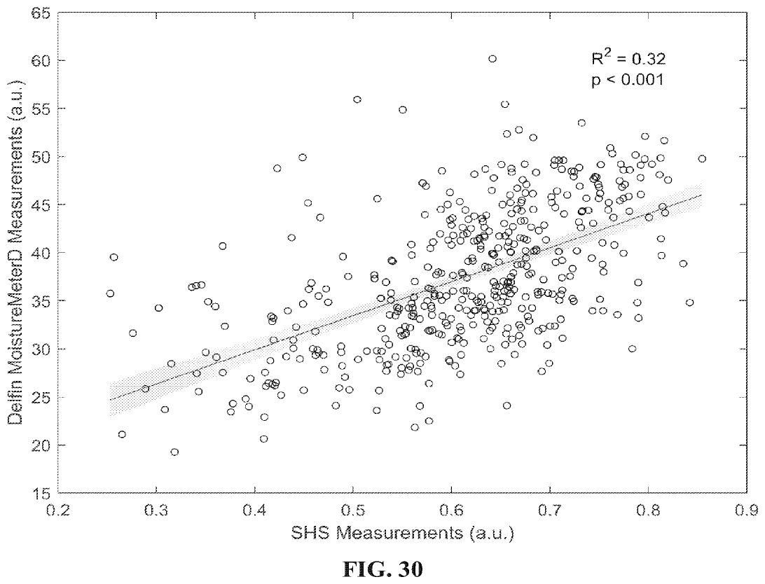

The mean ODS scores in subjects with AD were 1.6, 2.1, and 0.1 at the arm, leg, and forehead, respectively. The mean ODS scores in subjects without AD at the arm, leg, and forehead were all slightly lower at 0.7, 1.5, and 0, respectively. Median values at ODS 0 between subjects with and without AD were 0.6781 vs 0.6070 at the arm (p=0.09), 0.7224 vs 0.6501 at the leg (p=0.48), and 0.6940 vs 0.6882 at the forehead (p=0.94). The absolute difference of means at ODS 0 between subjects with vs. without AD were 0.0843 (14% decrease from HNL), 0.0773 (12%), and 0.0148 (2%) at the arm, leg, and forehead. P values from a Wilcoxon rank sum test comparing SHS measurements taken from ODS 0 with vs. without AD at the arm, leg, and forehead were 0.006 (n=4 vs 62, with vs. without AD), 0.08 (n=2 vs. 35), and 0.73 (n=17 vs. 125). While not a primary objective of the study, measurements of the SHS correlated with corneometers readings (r²=0.32, p<0.001), as shown in FIG. 30.

### DISCUSSIONS

There is a need for more objective outcome measures across the field of dermatology. Given the importance of skin barrier function to the pathogenesis of a wide range of skin diseases, there is a specific need for assessment tools in this area. Both AD and XC are conditions that have complex and varying courses with nuances that are likely underappreciated when solely evaluated with subjective scoring. While subjective measures and patient reported outcomes have value, objective digital biomarkers offer the opportunity to evaluate, compare, and detect changes in disease severity in a multitude of contexts from triggers to treatment without memory bias or inter-reviewer bias. With more reliable measurements comes the ability to derive more powerful and definitive insights from trials and follow up in clinical management. Additionally, as the fund of knowledge surrounding the skin barrier evolves and becomes more refined, the tools available for clinical evaluation of the skin barrier need to evolve in tandem. The Harmonising Outcome Measures for Eczema (HOME) initiative has previously established recommendations for measurement instruments such as the EASI and SCORAD to be standardized across trials. However, there is a need for biophysical measurements beyond physician-scales that offers more sensitivity, reliability, and scalability.

Previous work has already demonstrated the consistency of SHS measurements. In the current study, the SHS illustrates the ability of the system to provide reliable measurements that correlate with subjective clinical assessment tools, such as the ODS scoring system, across a spectrum of dry skin severity, as seen in FIG. 28. The findings with the SHS are also consistent with known variations in hydration across different locations, namely a higher hydration content at the forehead when compared to the arm and leg. The SHS is also capable of quantifying the changes in hydration seen with moisturizer application and yields results consistent with previous evidence that moisturizer use results in more significant hydration improvement in more severe dry skin.

Consideration must be given to areas of skin that have discordant ODS scores and SHS measurements, seen as outliers in FIG. 28. Here, the limitations of the ODS score as a mean of assessing dry skin severity are likely contributory. The ODS scoring system was designed by expert consensus and while it has been used in several studies, the system itself has not been definitively linked to skin barrier function or the progression of dry skin severity. It is also possible that the most superficial layers of the stratum corneum result in scale on visual inspection, but the skin barrier itself is intact.

Additionally, the novel use of dermoscopy to improve the ODS scoring system may be a more reliable and consistent means of scoring than general examination. The use of dermoscopy in this study enhanced the level of detail in the images and consistency in lighting and skin surface area. These attributes also allowed the study authors to achieve greater consistency in applying the ODS scoring system.

Looking at the effects of moisturizer, the role of absorption time must be considered to address the concern of measuring a layer of moisturizer. The possibility that a thin layer of moisturizer was retained on the surface of the skin and impacted subsequent measurements in our study cannot be definitively ruled out. However, the protocol was informed by a previous study that established moisturizer absorption time to be less than 10 minutes by infrared imaging and the increase in skin hydration after moisturizer application is consistent with previous findings as well.

In the study, it is important to note that the definition of healthy normal and xerotic skin is not an exact representation of healthy normal versus xerotic skin as seen clinically. Since XC is a clinical diagnosis that does not require assignment of an overall dry skin score, it is possible that there are patients who have been diagnosed with xerosis cutis but would not meet our criteria and vice versa. Here again, the limitations of the ODS scoring system must be acknowledged, especially the issues of repeatability and inter-reviewer reliability. Additionally, in the comparison of healthy normal and atopic skin, our study did not further stratify the AD group by ODS due to a limited number of AD subjects (n=18).

### CONCLUSIONS

The SHS offers benefits of small size, portability, accessibility, reliability, and consistency compared to existing devices used for research purposes. When compared specifically with traditional impedance devices, the use of thermal conductivity allows the SHS to take measurements across a greater range of tissue depths. Reliance on thermal conductivity rather than electrical impedance is also a likely reason that the SHS can be encapsulated without requiring direct contact between electronics components and the skin, resulting in greater flexibility and patient comfort. Considering practicality, the Bluetooth® capability and external data storage of the SHS means that users can easily organize their data on their own personal mobile device enabling home use. Given these considerations, there are several potential uses for the device as a diagnostic support tool, for longitudinal measurements to detect changes in disease severity and/or treatment response, and as an objective measure of efficacy that would allow for cross-comparison in clinical trials.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

1. H. W. Lim et al., The burden of skin disease in the United States. J Am Acad Dermatol 76, 958-972 e952 (2017).
2. B. A. Lowell et al., Dermatology in primary care: Prevalence and patient disposition. J Am Acad Dermatol 45, 250-255 (2001).
3. T. Basarab et al., Diagnostic accuracy and appropriateness of general practitioner referrals to a dermatology out-patient clinic. Br J Dermatol 135, 70-73 (1996).
4. R. F. Wagner et al., Diagnoses of skin disease: dermatologists vs. nondermatologists. J Dermatol Surg Oncol 11, 476-479 (1985).
5. A. R. Mulick et al., Classifying atopic dermatitis: protocol for a systematic review of subtypes (phenotypes) and associated characteristics. BMJ Open 8, e023097 (2018).
6. K. A. Walters, M. S. Roberts, Dermatologic, cosmeceutic, and cosmetic development: therapeutic and novel approaches. (Informa Healthcare, New York, 2008), pp. xi, 628 p.
7. S. R. Madhvapathy et al., Epidermal Electronic Systems for Measuring the Thermal Properties of Human Skin at Depths of up to Several Millimeters. Advanced Functional Materials 28, 1802083 (2018).
8. S. R. Krishnan et al., Wireless, Battery-Free Epidermal Electronics for Continuous, Quantitative, Multimodal Thermal Characterization of Skin. Small 14, e1803192 (2018).
9. R. C. Webb et al., Ultrathin conformal devices for precise and continuous thermal characterization of human skin. Nat Mater 12, 938-944 (2013).
10. S. E. Gustafsson, Transient plane source techniques for thermal conductivity and thermal diffusivity measurements of solid materials. Review of Scientific Instruments 62, 797-804 (1991).
11. K. E. Crawford et al., Advanced approaches for quantitative characterization of thermal transport properties in soft materials using thin, conformable resistive sensors. Extreme Mech Lett 22, 27-35 (2018).
12. S. Krishnan et al., Multimodal epidermal devices for hydration monitoring. Microsystems & Nanoengineering 3, 17014 (2017).
13. R. C. Webb et al., Thermal transport characteristics of human skin measured in vivo using ultrathin conformal arrays of thermal sensors and actuators. PLoS One 10, e0118131 (2015).
14. Y. Lee, K. Hwang, Skin thickness of Korean adults. Surgical and Radiologic Anatomy 24, 183-189 (2002).
15. E. Behrens, Thermal Conductivities of Composite Materials. J Compos Mater 2, 2-17 (1968).
16. M. Picardo et al., A. Mastrofrancesco, Sebaceous gland lipids. Dermatoendocrinol 1, 68-71 (2009).
17. N. S. Bogatishcheva et al., Critical properties, heat capacities, and thermal diffusivities of four saturated triglycerides. J Chem Thermodyn 113, 308-314 (2017).
18. V. Oreopoulou et al., 52 Frying of Foods. Handbook of Industrial Drying, Fourth Edition, (2006).
19. G. Wang et al., Measurement on the thermal conductivity of five saturated fatty acid ethyl esters components of biodiesel. Fluid Phase Equilibr 473, 106-111 (2018).
20. I. o. Medicine, Dietary Reference Intakes for Water, Potassium, Sodium, Chloride, and Sulfate. (The National Academies Press, Washington, D C, 2005), pp. 638.
21. N. Nakagawa et al., In vivo measurement of the water content in the dermis by confocal Raman spectroscopy. Skin Res Technol 16, 137-141 (2010).
22. E. E. Grinich et al., Validation of a novel smartphone application-enabled, patient-operated skin barrier device. Skin Res Technol 25, 612-617 (2019).
23. L. Ye et al., Validation of GPSkin Barrier® for assessing epidermal permeability barrier function and stratum corneum hydration in humans. Skin Res Technol 25, 25-29 (2019).
24. J. W. Fluhr et al., Comparative study of five instruments measuring stratum corneurn hydration (Comeometer CM 820 and CM 825, Skicon 200, Nova DPM 9003, DermaLab). Part I. In vitro. Skin Research and Technology 5, 161-170 (1999).
25. E. Alanen et al., Measurement of hydration in the stratum corneum with the MoistureMeter and comparison with the Corneometer. Skin Res Technol 10, 32-37 (2004).
26. R. Berke et al., Atopic dermatitis: an overview. Am Fam Physician 86, 35-42 (2012).
27. S. P. DaVeiga, Epidemiology of atopic dermatitis: a review. Allergy Asthma Proc 33, 227-234 (2012).
28. E. A. Brezinski et al., Economic Burden of Psoriasis in the United States A Systematic Review. Jama Dermatology 151, 651-658 (2015).
29. R. S. Stern et al., Psoriasis is common, carries a substantial burden even when not extensive, and is associated with widespread treatment dissatisfaction. J Invest Derm Symp P 9, 136-139 (2004).
30. F. F. Ferri, Ferri's Clinical Advisor 2019 E-Book: 5 Books in 1. (Elsevier Health Sciences, 2018).
31. Y. J. Li et al., Thin, Millimeter Scale Fingernail Sensors for Thermal Characterization of Nail Bed Tissue. Advanced Functional Materials 28, 1801380 (2018).
32. P. T. Tsilingiris, Thermophysical and transport properties of humid air at temperature range between 0 and 100 degrees C. Energ Convers Manage 49, 1098-1110 (2008).
33. https://product.tdk.com/info/en/catalog/datasheets/503021/tpd_commercial_ntc-thermistor_ntcg_en.pdf.
34. https://www.ti.com/lit/ug/slau603b/slau603b.pdf.

1. A hydration sensor, comprising:
a top layer for thermal, chemical and mechanical isolation of the hydration sensor from an environment;
a bottom layer operably placed on a target area of interest on skin; and
a flexible printed circuit board (f-PCB) disposed between the top layer and the bottom layer, wherein the f-PCB contains electronics for sensing and wireless communication, and the bottom layer operably serves as a direct interface between the f-PCB and the skin and comprises a flexible adhesive for attaching the hydration sensor to the skin.

2. The hydration sensor of claim 1, wherein the electronics comprises:
a heating circuit comprising a heating element for operably heating the target area of interest of the skin; and
a sensing circuit comprising a temperature sensor for simultaneously recording a transient temperature change (ΔT) thereof.

3. The hydration sensor of claim 2, wherein the heating element comprises a heater comprising at least one resistor.

4. The hydration sensor of claim 3, wherein the heater comprises two or more surface-mount (SMT) thin film resistors, thick film resistors, through-hole resistors, and/or ultrathin-film metal resistors connected in series to form a heater.

5. The hydration sensor of claim 2, wherein the temperature sensor comprises an SMT negative temperature coefficient thermistor, positive temperature coefficient thermistor, resistance temperature detector (RTD), thermocouple or any other conductive temperature sensor.

6. The hydration sensor of claim 1, wherein the heating element and the temperature sensor are arranged from each other by a distance.

7. The hydration sensor of claim 6, wherein the distance is determined by the design requirement of depth sensitivity into the skin, and ranges from about 10 μm to about 10 mm.

8. The hydration sensor of claim 6, wherein the heater and temperature sensor are the same component, wherein the distance between them is zero.

9. The hydration sensor of claim 2, wherein the sensing circuit further comprises a microcontroller (μC).

10. The hydration sensor of claim 9, wherein the heater is operably switchable between ON and OFF controlled by the microcontroller.

11. The hydration sensor of claim 9, wherein the microcontroller is programmable with custom-designed embedded codes using at least one of near field communication (NFC), Wi-Fi/Internet, Bluetooth, Bluetooth low energy (BLE), and Cellular communication protocols for wireless communication of the hydration sensor to a custom smartphone application.

12. The hydration sensor of claim 11, wherein the electronics further comprises a primary antenna tuned to primary frequency, and a secondary antenna.

13. The hydration sensor of claim 12, wherein the primary antenna is a transmission coil of an external device that is capable of wireless communications using the NFC protocol.

14. The hydration sensor of claim 13, wherein the external device is a smartphone, a tablet, computer or any electronic device with data reading capability.

15. The hydration sensor of claim 13, wherein the secondary antenna comprises a first antenna electronically connected to the microcontroller for powering the sensing circuit and for communicating data to the external device using the NFC protocol, and a second antenna electronically connected to the heating circuit for powering the heating element.

16. The hydration sensor of claim 15, wherein the first antenna and the second antenna are arranged in a concentric geometry.

17. The hydration sensor of claim 16, wherein the first antenna has a quality-factor (Q) that is relatively high to enable good communication distance and coupling across external devices with different primary antennae, and the second antenna has the quality-factor that is relatively low to support adequate power harvesting despite the difference between its resonance frequency and that of the primary coil.

18. The hydration sensor of claim 17, wherein the quality-factor of the first antenna is about 11, and the quality-factor of the second antenna is about 8.

19. The hydration sensor of claim 15, wherein the first antenna is tuned to the primary frequency, and the second antenna is tuned to a secondary frequency that is different from the primary frequency so as to prevent interference with the first antenna.

20. The hydration sensor of claim 19, wherein the primary frequency is a standard NFC frequency of about 13.56 MHz, and the secondary frequency is about 19.04 MHz.

21. The hydration sensor of claim 1, being operated using a single antenna.

22. The hydration sensor of claim 1, wherein the electronics comprises a generic design including blocks of:
(a) a powering system comprising voltage/power regulators driven by an external battery or magnetic induction to supply power to the heater, sensing and communication circuits;
(b) an analog to digital converters (ADC) chip or data modulator to prepare the output of the sensing circuit for transmission to an external readout device; and
(c) a transceiver chip having at least one of NFC, Wi-Fi/Internet, Bluetooth, BLE, and Cellular communication protocols to transmit the output signal to the external readout device.

23. The hydration sensor of claim 1, having a dynamic temperature range of about 18-45° C., adjustable through an amplifier gain, with a minimum resolution of about 15 mK, limited by the ADC.

24. The hydration sensor of claim 1, wherein the f-PCB is formed of a flexible material.

25. The hydration sensor of claim 24, wherein the flexible material comprises polyimide (PI), polyethylene terephthalate (PET), or any one of them in combination with a stiff PCB material including FR-4.

26. The hydration sensor of claim 25, wherein the f-PCB comprises open spaces and/or mechanical relief cuts for enhancing the overall flexibility, and limiting thermal transport through the PI, away from the sensing components.

27. The hydration sensor of claim 1, wherein the bottom layer comprises a flexible adhesive layer bonding to a thin layer of SiO₂ coated on a backside of the f-PCB.

28. The hydration sensor of claim 1, wherein the bottom layer adheres to the f-PCB through use of silicone bonding material, epoxy, glue, or commercial adhesive.

29. The hydration sensor of claim 27, wherein the bottom layer further comprises an ultrathin fabric of fiberglass or a reinforcement material embedded in the flexible adhesive layer for enhancing mechanical robustness of the hydration sensor.

30. The hydration sensor of claim 29, wherein the reinforcement material is flexible and has varying mesh density and thickness to lend tear resistance to the bottom layer.

31. The hydration sensor of claim 29, wherein the flexible adhesive layer is formed of silicone or silicone gel, or commercially available double-sided skin-safe adhesives, with the ratio of silicone and silicone gel being adjusted to co-optimize mechanical integrity and tackiness of the adhesive.

32. The hydration sensor of claim 1, wherein the top layer is a shell-like top encapsulation layer including small air gaps for thermally, mechanically, and chemically insulating the critical sensing components.

33. The hydration sensor of claim 1, wherein the top layer comprises:
an air pocket/gap/shell which thermally isolates the sensing area of the hydration sensor from the environment, thereby improving hydration sensor sensitivity;
a shell made of silicone or similar materials to chemically and mechanically isolate and protect the underlying layers of the hydration sensor from external elements including water, dust, and/or from user touch; or
a hollow shell-like structure to provide a soft touch/feel to the user.

34. The hydration sensor of claim 32, wherein the top layer is fabricated using molds to cure the silicone or similar materials including hypoallergenic.

35. The hydration sensor of claim 32, wherein the top layer is formed of a flexible material including silicone or silicone gel, low/high density polyethylene (LDPE/HDPE), polystyrene, and other flexible polymers.

36. The hydration sensor of claim 1, having low flexural rigidity and effective modulus, to facilitate application even on highly curved features of the skin.

37. The hydration sensor of claim 1, being compatible for use in conjunction with other adhesives/tapes/bandages for applications on highly curved features on the human body/skin.

38. The hydration sensor of claim 1, being a soft, thin, wireless, and battery-free sensor.

39. The hydration sensor of claim 2, wherein thermal properties of the skin comprise thermal conductivity (k) and thermal diffusivity (α) of the skin that are related to water content (φ) of the skin, wherein the water content is a function of a skin depth.

40. The hydration sensor of claim 39, wherein the water content φ_{E} (epidermis) and φ_{D} (dermis) are determined from the measured temperature change ΔT vs. time t.

41. The hydration sensor of claim 40, wherein the temperature change (ΔT) in the temperature sensor is operably captured by an ADC and transmitted to the external device.

42. The hydration sensor of claim 40, wherein the water content φ_{E} (epidermis), φ_{D}(dermis), and skin surface temperature T₀ are used to determine a normal state or a disease state of the skin.

43. The hydration sensor of claim 42, wherein the water content φ_{E} (epidermis), φ_{D}(dermis), and the skin surface temperature T₀ are used to diagnose various skin diseases.

44. The hydration sensor of claim 43, wherein the water content φ_{E} (epidermis), φ_{D}(dermis), and the skin surface temperature T₀ serve as quantitative metrics of an efficacy of a treatment of a skin disease, or other health and wellness products including skin moisturizers, lotions, and/or creams.

45. The hydration sensor of claim 40, wherein the aggregate water content φ comprising the full measurement volume serves as a quantitative metric for diagnosis of skin diseases or of an efficacy of a treatment of a skin disease.

46. The hydration sensor of claim 1, being usable for monitoring the skin condition.

47. The hydration sensor of claim 1, being usable for delivering treatment, monitoring the effects, modulating the treatment protocol as necessary, and/or potentially predicting for flares based on quantitative, individualized measurements on specific lesion sites.

48. The hydration sensor of claim 1, being usable for monitoring water content of internal organs for various diseases where traditional monitoring techniques fail to offer continuous assessment of organ health.

49. The hydration sensor of claim 1, being usable for monitoring organs during organ transport for applications in organ transplant.

50. The hydration sensor of claim 1, being usable in both clinical and at-home settings.

51. The hydration sensor of claim 1, wherein the skin health data (hydration of dermis, epidermis and skin temperature) for both lesions/healthy sites are shareable with physicians for remote medical care applications.

52. The hydration sensor of claim 1, being usable for applications to measure thermal conductivity, thermal diffusivity, heat capacity and other thermal properties of any material as a function of depth.

53. The hydration sensor of claim 1, being usable for applications to measure water content of any material surface as a function of depth, including hydrogels, plants (irrigation and agriculture applications), food preservation (dried food products, grains, fruits, meats), and/or concrete (industrial applications).

54. The hydration sensor of claim 1, being usable for monitoring composition of food/beverages, medicines/industrial chemicals.

55. The hydration sensor of claim 1, being re-usable and removal without irritation to the skin or damage to the hydration sensor.

56. The hydration sensor of claim 1, being compatible with alcohol-based cleaning wipes allowing for re-use across different users, without any damage to the hydration sensor or loss in efficacy of the hydration sensor adhesive.

57. The hydration sensor of claim 1, being sterilizable using alcohol, autoclave steam sterilization, and gas phase sterilization.

58-100. (canceled)
